SEQUENCE LISTING (167 sequences)

SEQ ID NO: 1 - DNA, 60 bp, Homo sapiens
  source 1..60: mol_type = genomic DNA
tttttaggggtctgcgcgctctcgcctggctgctgggaaggagggctgggaggcgctgaa

SEQ ID NO: 2 - DNA, 140 bp, Homo sapiens
  source 1..140: mol_type = genomic DNA
tccaggcgcgccgaccgctcaagcgctccaggtccacccggcggagggcagagaaagcgcgaccgcgcggcccgcagggttgcaagaagaaaacgagtgttatataatgagtctcagtggttgctcacaatgccaggcgc

SEQ ID NO: 3 - DNA, 60 bp, Homo sapiens
  source 1..60: mol_type = genomic DNA
ggctgataccagagaggacccggagcgcgaaccagaggttcgacctccagggcagcgcag

SEQ ID NO: 4 - DNA, 130 bp, Homo sapiens
  source 1..130: mol_type = genomic DNA
ggggcgccaaatgcccacgtgttgatgaaaccagtgagatgggaacaggcggcgggaaaccagacagaggaagagctagggaggagaccccagccccggatcctgggtcgccagggttttccgcgcgcat

SEQ ID NO: 5 - DNA, 70 bp, Homo sapiens
  source 1..70: mol_type = genomic DNA
tctgagaagtgtcctcctcgctctcttataaaaacaggacttgttgccgaggtcagcgcgcgcatcgagt

SEQ ID NO: 6 - DNA, 130 bp, Homo sapiens
  source 1..130: mol_type = genomic DNA
atcagcgcgaactattcgtttagtggccttaaaacaccctggtttcaccctcagctattttcaagttcccgtgtgcctggcactttctccgtgcgagaagcaccggagggtgcggacgcgccacagtctg

SEQ ID NO: 7 - DNA, 150 bp, Homo sapiens
  source 1..150: mol_type = genomic DNA
tgctctgcgcagcgtggagggcaacgggactgggaggcggcttctgccgccgggcactcgcttccaggtggcttacgaggattcagactgtgggaaccgtgcgctcttcagcttggtggaccgcgtggcggcggcgcggggcgccaagcc

SEQ ID NO: 8 - DNA, 70 bp, Homo sapiens
  source 1..70: mol_type = genomic DNA
cgcgcagaactttgcggtggcgcttagcgcctcctttgcccagacccttcccgcctttgccgcgcccaga

SEQ ID NO: 9 - DNA, 70 bp, Homo sapiens
  source 1..70: mol_type = genomic DNA
cgtaatcgccggtgtaactcatgttggctggggggcctcccggcgcgcgcggagaggctggggtgcgccc

SEQ ID NO: 10 - DNA, 60 bp, Homo sapiens
  source 1..60: mol_type = genomic DNA
cgattcagagggccccggtcggagctgtcggagattgagcgcgcgcggtcccgggatctc

SEQ ID NO: 11 - DNA, 140 bp, Homo sapiens
  source 1..140: mol_type = genomic DNA
cgcgcggcagccctccgtgcgcgcaggctcgggtgcgttgttcgcgggggtgaattgtgaagaaccatcgcggggtccttcctgctgaggccgcggacaccgtgacctcgctgctctgggtctgcagggaaacgtaggaa

SEQ ID NO: 12 - DNA, 100 bp, Homo sapiens
  source 1..100: mol_type = genomic DNA
tgacctttaggggctgttactctcagaccaggcccagcagcacccggcgcatttacgtcggatctgacccctgcaagcaccggcgcgaccgcgctagcgg

SEQ ID NO: 13 - DNA, 70 bp, Homo sapiens
  source 1..70: mol_type = genomic DNA
agaacttcgtgggcaggtaagcgcgcagggcgcggagtaaggatgcggcagtggggcgaccccgctgcgg

SEQ ID NO: 14 - DNA, 70 bp, Homo sapiens
  source 1..70: mol_type = genomic DNA
ctgcctgcgctcactacgggttttttagttggcgccttaatgtttgtaacacttttagagcgcttgctct

SEQ ID NO: 15 - DNA, 140 bp, Homo sapiens
  source 1..140: mol_type = genomic DNA
ggccgggaagcgcggggtgagaaagcgaggtgggtggcgagagcgtgagcgcccctctgctgaccccggggagcgtggactacgagttggcgcccaagtccagaatccgcgcgcaccgcggtaagctgcgccttttgaaa

SEQ ID NO: 16 - DNA, 60 bp, Homo sapiens
  source 1..60: mol_type = genomic DNA
agactaacttttctcttgtacagaatcatcaggctaaatttttggcattatttcagtcct

SEQ ID NO: 17 - DNA, 80 bp, synthetic construct
  misc_feature 1..80: note = Control construct
  source 1..80: mol_type = other DNA
aagtctcgagtcgatgtcactaaccatgagtattccatgtgccgtcatctaacaagtactgcataccctggataatggct

SEQ ID NO: 18 - DNA, 100 bp, synthetic construct
  misc_feature 1..100: note = Control construct
  source 1..100: mol_type = other DNA
taccaggtctacaaagctcgactctgcctgcgctcactacgggttttttagttggcgccttaatgtttgtaacacttttagagcgcttgctctgatcctc

SEQ ID NO: 19 - DNA, 20 bp, synthetic construct
  misc_feature 1..20: note = Primer
  source 1..20: mol_type = other DNA
aagtctcgagtcgatgtcac

SEQ ID NO: 20 - DNA, 20 bp, synthetic construct
  misc_feature 1..20: note = Primer
  source 1..20: mol_type = other DNA
agccattatccagggtatgc

SEQ ID NO: 21 - DNA, 23 bp, synthetic construct
  misc_feature 1..23: note = Probe
  source 1..23: mol_type = other DNA
accatgagtattccatgtgccgt

SEQ ID NO: 22 - DNA, 22 bp, synthetic construct
  misc_feature 1..22: note = Primer
  source 1..22: mol_type = other DNA
taccaggtctacaaagctcgac

SEQ ID NO: 23 - DNA, 23 bp, synthetic construct
  misc_feature 1..23: note = Primer
  source 1..23: mol_type = other DNA
acgcctactaatacacatcggag

SEQ ID NO: 24 - DNA, 25 bp, synthetic construct
  misc_feature 1..25: note = Probe
  source 1..25: mol_type = other DNA
cgctcactacgggttttttagttgg

SEQ ID NO: 25 - DNA, 21 bp, synthetic construct
  misc_feature 1..21: note = Primer
  source 1..21: mol_type = other DNA
cagacccggttaacactgaag

SEQ ID NO: 26 - DNA, 23 bp, synthetic construct
  misc_feature 1..23: note = Primer
  source 1..23: mol_type = other DNA
gacccggttaacactgaagttgc

SEQ ID NO: 27 - DNA, 20 bp, synthetic construct
  misc_feature 1..20: note = Primer
  source 1..20: mol_type = other DNA
ccagacccggttaacactga

SEQ ID NO: 28 - DNA, 22 bp, synthetic construct
  misc_feature 1..22: note = Primer
  source 1..22: mol_type = other DNA
ccggttaacactgaagttgctt

SEQ ID NO: 29 - DNA, 21 bp, synthetic construct
  misc_feature 1..21: note = Primer
  source 1..21: mol_type = other DNA
tccagacccggttaacactga

SEQ ID NO: 30 - DNA, 20 bp, synthetic construct
  misc_feature 1..20: note = Primer
  source 1..20: mol_type = other DNA
ctcagatctgccctcgcttc

SEQ ID NO: 31 - DNA, 20 bp, synthetic construct
  misc_feature 1..20: note = Primer
  source 1..20: mol_type = other DNA
ctcagatctgccctcgcttc

SEQ ID NO: 32 - DNA, 23 bp, synthetic construct
  misc_feature 1..23: note = Primer
  source 1..23: mol_type = other DNA
gcaactcctacagacactcagat

SEQ ID NO: 33 - DNA, 20 bp, synthetic construct
  misc_feature 1..20: note = Primer
  source 1..20: mol_type = other DNA
gacactcagatctgccctcg

SEQ ID NO: 34 - DNA, 25 bp, synthetic construct
  misc_feature 1..25: note = Primer
  source 1..25: mol_type = other DNA
atcagaacaattagagagtaatgat

SEQ ID NO: 35 - DNA, 19 bp, synthetic construct
  misc_feature 1..19: note = Primer
  source 1..19: mol_type = other DNA
cagatctgccctcgcttca

SEQ ID NO: 36 - DNA, 20 bp, synthetic construct
  misc_feature 1..20: note = Primer
  source 1..20: mol_type = other DNA
ggtccagcagctagtccact

SEQ ID NO: 37 - DNA, 20 bp, synthetic construct
  misc_feature 1..20: note = Primer
  source 1..20: mol_type = other DNA
ctccctggtccagcagctag

SEQ ID NO: 38 - DNA, 18 bp, synthetic construct
  misc_feature 1..18: note = Primer
  source 1..18: mol_type = other DNA
cccgcctggctgctccag

SEQ ID NO: 39 - DNA, 21 bp, synthetic construct
  misc_feature 1..21: note = Primer
  source 1..21: mol_type = other DNA
caccaggtgcaaaagatgcct

SEQ ID NO: 40 - DNA, 20 bp, synthetic construct
  misc_feature 1..20: note = Primer
  source 1..20: mol_type = other DNA
cgggaagggaaaggccacat

SEQ ID NO: 41 - DNA, 21 bp, synthetic construct
  misc_feature 1..21: note = Primer
  source 1..21: mol_type = other DNA
aaggccacatcttcacgcctt

SEQ ID NO: 42 - DNA, 22 bp, synthetic construct
  misc_feature 1..22: note = Primer
  source 1..22: mol_type = other DNA
tgagcaaccactgagactcatt

SEQ ID NO: 43 - DNA, 19 bp, synthetic construct
  misc_feature 1..19: note = Primer
  source 1..19: mol_type = other DNA
cgtcctggagctccgactc

SEQ ID NO: 44 - DNA, 19 bp, synthetic construct
  misc_feature 1..19: note = Primer
  source 1..19: mol_type = other DNA
ccgactcccaccccatctg

SEQ ID NO: 45 - DNA, 20 bp, synthetic construct
  misc_feature 1..20: note = Primer
  source 1..20: mol_type = other DNA
ataccagagaggacccggag

SEQ ID NO: 46 - DNA, 21 bp, synthetic construct
  misc_feature 1..21: note = Primer
  source 1..21: mol_type = other DNA
gaaccagaggttcgacctcca

SEQ ID NO: 47 - DNA, 19 bp, synthetic construct
  misc_feature 1..19: note = Primer
  source 1..19: mol_type = other DNA
cccggcttcggagcgggaa

SEQ ID NO: 48 - DNA, 21 bp, synthetic construct
  misc_feature 1..21: note = Primer
  source 1..21: mol_type = other DNA
cagtttcaccgctcgatcttg

SEQ ID NO: 49 - DNA, 20 bp, synthetic construct
  misc_feature 1..20: note = Primer
  source 1..20: mol_type = other DNA
cgagtccagggcgaggtaag

SEQ ID NO: 50 - DNA, 21 bp, synthetic construct
  misc_feature 1..21: note = Primer
  source 1..21: mol_type = other DNA
gaggctgataccagagaggac

SEQ ID NO: 51 - DNA, 21 bp, synthetic construct
  misc_feature 1..21: note = Primer
  source 1..21: mol_type = other DNA
cacaactgcctgcgagaaaca

SEQ ID NO: 52 - DNA, 19 bp, synthetic construct
  misc_feature 1..19: note = Primer
  source 1..19: mol_type = other DNA
agcttgtggcgaccccgtc

SEQ ID NO: 53 - DNA, 21 bp, synthetic construct
  misc_feature 1..21: note = Primer
  source 1..21: mol_type = other DNA
tagctcatcccgtccagcttg

SEQ ID NO: 54 - DNA, 19 bp, synthetic construct
  misc_feature 1..19: note = Primer
  source 1..19: mol_type = other DNA
cgcctcagccactcctggg

SEQ ID NO: 55 - DNA, 20 bp, synthetic construct
  misc_feature 1..20: note = Primer
  source 1..20: mol_type = other DNA
ggccttggagcccaaaccag

SEQ ID NO: 56 - DNA, 19 bp, synthetic construct
  misc_feature 1..19: note = Primer
  source 1..19: mol_type = other DNA
gctcgggcagagccaagga

SEQ ID NO: 57 - DNA, 20 bp, synthetic construct
  misc_feature 1..20: note = Primer
  source 1..20: mol_type = other DNA
tcttgtgtgggtgccttgga

SEQ ID NO: 58 - DNA, 22 bp, synthetic construct
  misc_feature 1..22: note = Primer
  source 1..22: mol_type = other DNA
ctggaggtcgaacctctggttc

SEQ ID NO: 59 - DNA, 19 bp, synthetic construct
  misc_feature 1..19: note = Primer
  source 1..19: mol_type = other DNA
cgctccgaagccggggtac

SEQ ID NO: 60 - DNA, 20 bp, synthetic construct
  misc_feature 1..20: note = Primer
  source 1..20: mol_type = other DNA
ggtgggagtcggagctccag

SEQ ID NO: 61 - DNA, 20 bp, synthetic construct
  misc_feature 1..20: note = Primer
  source 1..20: mol_type = other DNA
ggagcgacccaaatgtggag

SEQ ID NO: 62 - DNA, 22 bp, synthetic construct
  misc_feature 1..22: note = Primer
  source 1..22: mol_type = other DNA
cctgggcatcgtttacatttgg

SEQ ID NO: 63 - DNA, 20 bp, synthetic construct
  misc_feature 1..20: note = Primer
  source 1..20: mol_type = other DNA
gcatcgtttacatttggggc

SEQ ID NO: 64 - DNA, 21 bp, synthetic construct
  misc_feature 1..21: note = Primer
  source 1..21: mol_type = other DNA
gagtagagcaccaagatagtg

SEQ ID NO: 65 - DNA, 20 bp, synthetic construct
  misc_feature 1..20: note = Primer
  source 1..20: mol_type = other DNA
gcagccgcaccttttgggat

SEQ ID NO: 66 - DNA, 20 bp, synthetic construct
  misc_feature 1..20: note = Primer
  source 1..20: mol_type = other DNA
gggatgggatggtttggaga

SEQ ID NO: 67 - DNA, 23 bp, synthetic construct
  misc_feature 1..23: note = Primer
  source 1..23: mol_type = other DNA
gagtctaacaaactaacctgatg

SEQ ID NO: 68 - DNA, 21 bp, synthetic construct
  misc_feature 1..21: note = Primer
  source 1..21: mol_type = other DNA
acaggacttgttgccgaggtc

SEQ ID NO: 69 - DNA, 23 bp, synthetic construct
  misc_feature 1..23: note = Primer
  source 1..23: mol_type = other DNA
atggacggaaataagcaaaagca

SEQ ID NO: 70 - DNA, 22 bp, synthetic construct
  misc_feature 1..22: note = Primer
  source 1..22: mol_type = other DNA
gcaaaagcaaaacaaccccttc

SEQ ID NO: 71 - DNA, 22 bp, synthetic construct
  misc_feature 1..22: note = Primer
  source 1..22: mol_type = other DNA
ttccgtgaaagcaatgacacag

SEQ ID NO: 72 - DNA, 22 bp, synthetic construct
  misc_feature 1..22: note = Primer
  source 1..22: mol_type = other DNA
aataaaacaaccgctgccgaca

SEQ ID NO: 73 - DNA, 21 bp, synthetic construct
  misc_feature 1..21: note = Primer
  source 1..21: mol_type = other DNA
caatgacacagcagaaaccac

SEQ ID NO: 74 - DNA, 20 bp, synthetic construct
  misc_feature 1..20: note = Primer
  source 1..20: mol_type = other DNA
gccaggcactcgctggatct

SEQ ID NO: 75 - DNA, 21 bp, synthetic construct
  misc_feature 1..21: note = Primer
  source 1..21: mol_type = other DNA
ccgccatcgataacagatcag

SEQ ID NO: 76 - DNA, 20 bp, synthetic construct
  misc_feature 1..20: note = Primer
  source 1..20: mol_type = other DNA
ggcaccgccatcgataacag

SEQ ID NO: 77 - DNA, 21 bp, synthetic construct
  misc_feature 1..21: note = Primer
  source 1..21: mol_type = other DNA
cccctaaacttagccagttcg

SEQ ID NO: 78 - DNA, 24 bp, synthetic construct
  misc_feature 1..24: note = Primer
  source 1..24: mol_type = other DNA
tggcaggaacatgaataataaatg

SEQ ID NO: 79 - DNA, 19 bp, synthetic construct
  misc_feature 1..19: note = Primer
  source 1..19: mol_type = other DNA
ggcggcggctcagactgtg

SEQ ID NO: 80 - DNA, 21 bp, synthetic construct
  misc_feature 1..21: note = Primer
  source 1..21: mol_type = other DNA
ggcttacgaggattcagactg

SEQ ID NO: 81 - DNA, 19 bp, synthetic construct
  misc_feature 1..19: note = Primer
  source 1..19: mol_type = other DNA
ggactgggaggcggcttct

SEQ ID NO: 82 - DNA, 19 bp, synthetic construct
  misc_feature 1..19: note = Primer
  source 1..19: mol_type = other DNA
cgtggagggcaacgggact

SEQ ID NO: 83 - DNA, 20 bp, synthetic construct
  misc_feature 1..20: note = Primer
  source 1..20: mol_type = other DNA
cactcgcttccaggtggctt

SEQ ID NO: 84 - DNA, 20 bp, synthetic construct
  misc_feature 1..20: note = Primer
  source 1..20: mol_type = other DNA
cggccatcgagtatgctctg

SEQ ID NO: 85 - DNA, 23 bp, synthetic construct
  misc_feature 1..23: note = Primer
  source 1..23: mol_type = other DNA
gatgactcgtacttgttttcact

SEQ ID NO: 86 - DNA, 21 bp, synthetic construct
  misc_feature 1..21: note = Primer
  source 1..21: mol_type = other DNA
cccaggataaggtctggcttg

SEQ ID NO: 87 - DNA, 21 bp, synthetic construct
  misc_feature 1..21: note = Primer
  source 1..21: mol_type = other DNA
ctgctgcatactcgcacactg

SEQ ID NO: 88 - DNA, 20 bp, synthetic construct
  misc_feature 1..20: note = Primer
  source 1..20: mol_type = other DNA
cacactggccccaggataag

SEQ ID NO: 89 - DNA, 19 bp, synthetic construct
  misc_feature 1..19: note = Primer
  source 1..19: mol_type = other DNA
gatgcaagccgggccactg

SEQ ID NO: 90 - DNA, 21 bp, synthetic construct
  misc_feature 1..21: note = Primer
  source 1..21: mol_type = other DNA
ggtgatgccctgaatgttgtc

SEQ ID NO: 91 - DNA, 19 bp, synthetic construct
  misc_feature 1..19: note = Primer
  source 1..19: mol_type = other DNA
cttaacgcctccacgccgt

SEQ ID NO: 92 - DNA, 18 bp, synthetic construct
  misc_feature 1..18: note = Primer
  source 1..18: mol_type = other DNA
ccgtgccaggcgtcggat

SEQ ID NO: 93 - DNA, 24 bp, synthetic construct
  misc_feature 1..24: note = Primer
  source 1..24: mol_type = other DNA
tttgtttagtacaagacatgtctg

SEQ ID NO: 94 - DNA, 23 bp, synthetic construct
  misc_feature 1..23: note = Primer
  source 1..23: mol_type = other DNA
cgtgttgcattttgtttagtaca

SEQ ID NO: 95 - DNA, 19 bp, synthetic construct
  misc_feature 1..19: note = Primer
  source 1..19: mol_type = other DNA
gctgcgaggcttgccgtgt

SEQ ID NO: 96 - DNA, 21 bp, synthetic construct
  misc_feature 1..21: note = Primer
  source 1..21: mol_type = other DNA
actcggtgttctcaccgaaag

SEQ ID NO: 97 - DNA, 21 bp, synthetic construct
  misc_feature 1..21: note = Primer
  source 1..21: mol_type = other DNA
acgtaatcgccggtgtaactc

SEQ ID NO: 98 - DNA, 22 bp, synthetic construct
  misc_feature 1..22: note = Primer
  source 1..22: mol_type = other DNA
cctttctttgtagccacctcag

SEQ ID NO: 99 - DNA, 21 bp, synthetic construct
  misc_feature 1..21: note = Primer
  source 1..21: mol_type = other DNA
ggaagcaacagatcgtcactc

SEQ ID NO: 100 - DNA, 22 bp, synthetic construct
  misc_feature 1..22: note = Primer
  source 1..22: mol_type = other DNA
ttgagcacaagcatgctgcatg

SEQ ID NO: 101 - DNA, 20 bp, synthetic construct
  misc_feature 1..20: note = Primer
  source 1..20: mol_type = other DNA
ccctctgcacactcgagaac

SEQ ID NO: 102 - DNA, 21 bp, synthetic construct
  misc_feature 1..21: note = Primer
  source 1..21: mol_type = other DNA
ggacgagagttgagctctcac

SEQ ID NO: 103 - DNA, 21 bp, synthetic construct
  misc_feature 1..21: note = Primer
  source 1..21: mol_type = other DNA
aggttgcagtgagccggaatg

SEQ ID NO: 104 - DNA, 25 bp, synthetic construct
  misc_feature 1..25: note = Primer
  source 1..25: mol_type = other DNA
aataccatatagtgaacacctaaga

SEQ ID NO: 105 - DNA, 24 bp, synthetic construct
  misc_feature 1..24: note = Primer
  source 1..24: mol_type = other DNA
ccaatttttgtgtttttagtagag

SEQ ID NO: 106 - DNA, 20 bp, synthetic construct
  misc_feature 1..20: note = Primer
  source 1..20: mol_type = other DNA
ggatccagggcgattcagag

SEQ ID NO: 107 - DNA, 19 bp, synthetic construct
  misc_feature 1..19: note = Primer
  source 1..19: mol_type = other DNA
cagggcctcgtcggagatc

SEQ ID NO: 108 - DNA, 19 bp, synthetic construct
  misc_feature 1..19: note = Primer
  source 1..19: mol_type = other DNA
tcgtcggagatcccgggac

SEQ ID NO: 109 - DNA, 21 bp, synthetic construct
  misc_feature 1..21: note = Primer
  source 1..21: mol_type = other DNA
tgctgcccagcttcttccaca

SEQ ID NO: 110 - DNA, 17 bp, synthetic construct
  misc_feature 1..17: note = Primer
  source 1..17: mol_type = other DNA
ggtcccgcggcctccag

SEQ ID NO: 111 - DNA, 17 bp, synthetic construct
  misc_feature 1..17: note = Primer
  source 1..17: mol_type = other DNA
gcagcttcgccgcccgg

SEQ ID NO: 112 - DNA, 18 bp, synthetic construct
  misc_feature 1..18: note = Primer
  source 1..18: mol_type = other DNA
cggaccggccaggggtcc

SEQ ID NO: 113 - DNA, 20 bp, synthetic construct
  misc_feature 1..20: note = Primer
  source 1..20: mol_type = other DNA
caggacccgctcagttccac

SEQ ID NO: 114 - DNA, 19 bp, synthetic construct
  misc_feature 1..19: note = Primer
  source 1..19: mol_type = other DNA
cagggatgtgagtgggcgg

SEQ ID NO: 115 - DNA, 20 bp, synthetic construct
  misc_feature 1..20: note = Primer
  source 1..20: mol_type = other DNA
gaagagggccgcaaaccaac

SEQ ID NO: 116 - DNA, 19 bp, synthetic construct
  misc_feature 1..19: note = Primer
  source 1..19: mol_type = other DNA
acccgccgcactgacaggt

SEQ ID NO: 117 - DNA, 22 bp, synthetic construct
  misc_feature 1..22: note = Primer
  source 1..22: mol_type = other DNA
tttcctacgtttccctgcagac

SEQ ID NO: 118 - DNA, 20 bp, synthetic construct
  misc_feature 1..20: note = Primer
  source 1..20: mol_type = other DNA
atcctgcccgctcctgacaa

SEQ ID NO: 119 - DNA, 20 bp, synthetic construct
  misc_feature 1..20: note = Primer
  source 1..20: mol_type = other DNA
ccagcaccaggagcgtgttc

SEQ ID NO: 120 - DNA, 22 bp, synthetic construct
  misc_feature 1..22: note = Primer
  source 1..22: mol_type = other DNA
cgcgatggttcttcacaattca

SEQ ID NO: 121 - DNA, 21 bp, synthetic construct
  misc_feature 1..21: note = Primer
  source 1..21: mol_type = other DNA
acgcgacgctctaaaggaagt

SEQ ID NO: 122 - DNA, 20 bp, synthetic construct
  misc_feature 1..20: note = Primer
  source 1..20: mol_type = other DNA
ctctcagaccaggcccagca

SEQ ID NO: 123 - DNA, 20 bp, synthetic construct
  misc_feature 1..20: note = Primer
  source 1..20: mol_type = other DNA
gctgaggcaaagcgactcca

SEQ ID NO: 124 - DNA, 22 bp, synthetic construct
  misc_feature 1..22: note = Primer
  source 1..22: mol_type = other DNA
ctctaaaggaagtgacctttag

SEQ ID NO: 125 - DNA, 20 bp, synthetic construct
  misc_feature 1..20: note = Primer
  source 1..20: mol_type = other DNA
ggctgttactctcagaccag

SEQ ID NO: 126 - DNA, 21 bp, synthetic construct
  misc_feature 1..21: note = Primer
  source 1..21: mol_type = other DNA
aacctaccgactgacctgcat

SEQ ID NO: 127 - DNA, 21 bp, synthetic construct
  misc_feature 1..21: note = Primer
  source 1..21: mol_type = other DNA
cctgcatgacgtaggtccact

SEQ ID NO: 128 - DNA, 20 bp, synthetic construct
  misc_feature 1..20: note = Primer
  source 1..20: mol_type = other DNA
gggtcagatccgacgtaaag

SEQ ID NO: 129 - DNA, 19 bp, synthetic construct
  misc_feature 1..19: note = Primer
  source 1..19: mol_type = other DNA
gcgtgttcccagccgctag

SEQ ID NO: 130 - DNA, 20 bp, synthetic construct
  misc_feature 1..20: note = Primer
  source 1..20: mol_type = other DNA
gggattggccgagtcaggtc

SEQ ID NO: 131 - DNA, 20 bp, synthetic construct
  misc_feature 1..20: note = Primer
  source 1..20: mol_type = other DNA
acagagctgcctgctggagc

SEQ ID NO: 132 - DNA, 19 bp, synthetic construct
  misc_feature 1..19: note = Primer
  source 1..19: mol_type = other DNA
cggcaagagctcggtgctc

SEQ ID NO: 133 - DNA, 21 bp, synthetic construct
  misc_feature 1..21: note = Primer
  source 1..21: mol_type = other DNA
agaacttcgtgggcaggtaag

SEQ ID NO: 134 - DNA, 21 bp, synthetic construct
  misc_feature 1..21: note = Primer
  source 1..21: mol_type = other DNA
cgtctgcaggaccgcgttttc

SEQ ID NO: 135 - DNA, 19 bp, synthetic construct
  misc_feature 1..19: note = Primer
  source 1..19: mol_type = other DNA
agctgatcccgctggtgaa

SEQ ID NO: 136 - DNA, 22 bp, synthetic construct
  misc_feature 1..22: note = Primer
  source 1..22: mol_type = other DNA
aatggctttgtatcgcacgatg

SEQ ID NO: 137 - DNA, 20 bp, synthetic construct
  misc_feature 1..20: note = Primer
  source 1..20: mol_type = other DNA
ccactgccgcatccttactc

SEQ ID NO: 138 - DNA, 20 bp, synthetic construct
  misc_feature 1..20: note = Primer
  source 1..20: mol_type = other DNA
cctggtccatccaccctctg

SEQ ID NO: 139 - DNA, 19 bp, synthetic construct
  misc_feature 1..19: note = Primer
  source 1..19: mol_type = other DNA
tgcccgtccacgttccaac

SEQ ID NO: 140 - DNA, 19 bp, synthetic construct
  misc_feature 1..19: note = Primer
  source 1..19: mol_type = other DNA
cacggacaccgccactctg

SEQ ID NO: 141 - DNA, 21 bp, synthetic construct
  misc_feature 1..21: note = Primer
  source 1..21: mol_type = other DNA
gagcattggcacccctgattc

SEQ ID NO: 142 - DNA, 21 bp, synthetic construct
  misc_feature 1..21: note = Primer
  source 1..21: mol_type = other DNA
cttacccgtggtttctgcctg

SEQ ID NO: 143 - DNA, 21 bp, synthetic construct
  misc_feature 1..21: note = Primer
  source 1..21: mol_type = other DNA
agagcattggcacccctgatt

SEQ ID NO: 144 - DNA, 22 bp, synthetic construct
  misc_feature 1..22: note = Primer
  source 1..22: mol_type = other DNA
acttctcttacccgtggtttct

SEQ ID NO: 145 - DNA, 19 bp, synthetic construct
  misc_feature 1..19: note = Primer
  source 1..19: mol_type = other DNA
ctggggcctacgcgaagct

SEQ ID NO: 146 - DNA, 25 bp, synthetic construct
  misc_feature 1..25: note = Primer
  source 1..25: mol_type = other DNA
ctctaaaagtgttacaaacattaag

SEQ ID NO: 147 - DNA, 20 bp, synthetic construct
  misc_feature 1..20: note = Primer
  source 1..20: mol_type = other DNA
tggaatcgtgcccggatcag

SEQ ID NO: 148 - DNA, 26 bp, synthetic construct
  misc_feature 1..26: note = Primer
  source 1..26: mol_type = other DNA
tttttggaacgttaaataatttccta

SEQ ID NO: 149 - DNA, 22 bp, synthetic construct
  misc_feature 1..22: note = Primer
  source 1..22: mol_type = other DNA
cctcagtttcctcaaaaggaat

SEQ ID NO: 150 - DNA, 20 bp, synthetic construct
  misc_feature 1..20: note = Primer
  source 1..20: mol_type = other DNA
cgtgcccggatcagagcaag

SEQ ID NO: 151 - DNA, 20 bp, synthetic construct
  misc_feature 1..20: note = Primer
  source 1..20: mol_type = other DNA
gcctggaatcgtgcccggat

SEQ ID NO: 152 - DNA, 23 bp, synthetic construct
  misc_feature 1..23: note = Primer
  source 1..23: mol_type = other DNA
tccaaggaagactaaaaacccag

SEQ ID NO: 153 - DNA, 21 bp, synthetic construct
  misc_feature 1..21: note = Primer
  source 1..21: mol_type = other DNA
taaaaacccaggccgggaagc

SEQ ID NO: 154 - DNA, 22 bp, synthetic construct
  misc_feature 1..22: note = Primer
  source 1..22: mol_type = other DNA
tggtttgttccaaggagtacag

SEQ ID NO: 155 - DNA, 23 bp, synthetic construct
  misc_feature 1..23: note = Primer
  source 1..23: mol_type = other DNA
agtacagatagccttttcaaaag

SEQ ID NO: 156 - DNA, 23 bp, synthetic construct
  misc_feature 1..23: note = Primer
  source 1..23: mol_type = other DNA
agcaaggtgaagactaacttttc

SEQ ID NO: 157 - DNA, 23 bp, synthetic construct
  misc_feature 1..23: note = Primer
  source 1..23: mol_type = other DNA
cttgtacagaatcatcaggctaa

SEQ ID NO: 158 - DNA, 25 bp, synthetic construct
  misc_feature 1..25: note = Primer
  source 1..25: mol_type = other DNA
gtccttggagacatctgagagattc

SEQ ID NO: 159 - DNA, 20 bp, synthetic construct
  misc_feature 1..20: note = Primer
  source 1..20: mol_type = other DNA
gacatctgagagattccggg

SEQ ID NO: 160 - DNA, 22 bp, synthetic construct
  misc_feature 1..22: note = Primer
  source 1..22: mol_type = other DNA
caaagaaagcaaggtgaagact

SEQ ID NO: 161 - DNA, 24 bp, synthetic construct
  misc_feature 1..24: note = Primer
  source 1..24: mol_type = other DNA
cagtccttggagacatctgagaga

SEQ ID NO: 162 - DNA, 23 bp, synthetic construct
  misc_feature 1..23: note = Primer
  source 1..23: mol_type = other DNA
tgaagtgaactattccttaggtg

SEQ ID NO: 163 - DNA, 23 bp, synthetic construct
  misc_feature 1..23: note = Primer
  source 1..23: mol_type = other DNA
gtctccaaggactgaaataatgc

SEQ ID NO: 164 - DNA, 28 bp, synthetic construct
  misc_feature 1..28: note = Primer
  source 1..28: mol_type = other DNA
gagtagcaaaaaatagctgaagtgaact

SEQ ID NO: 165 - DNA, 23 bp, synthetic construct
  misc_feature 1..23: note = Primer
  source 1..23: mol_type = other DNA
tgacaaccaatgagtagcaaaaa

SEQ ID NO: 166 - DNA, 20 bp, synthetic construct
  misc_feature 1..20: note = Primer
  source 1..20: mol_type = other DNA
gttgtcagtgtggccagaga

SEQ ID NO: 167 - DNA, 21 bp, synthetic construct
  misc_feature 1..21: note = Primer
  source 1..21: mol_type = other DNA
tgttgtcagtgtggccagaga

(19) United States
(12) Patent Application Publication
(10) Pub. No.: US 2026/0209859 A1
(43) Pub. Date: Jul. 23, 2026

(54) ASSESSMENT OF HEMATURIA AND OTHER URINARY TRACT SYMPTOMS

(71) Applicant: NUCLEIX LTD., Rehovot (IL)

(72) Inventors: Danny FRUMKIN, Rehovot (IL); Adam WASSERSTROM, Ness Ziona (IL); Aharona SHUALI, Tel Aviv (IL); Shmuel ADLER, Givat Shmuel (IL)

(73) Assignee: NUCLEIX LTD., Rehovot (IL)

(21) Appl. No.: 19/158,187

(22) PCT Filed: Feb. 21, 2023
(86) PCT No.: PCT/IL2023/050181

§ 371 Date: Aug. 20, 2025

(51) Int. Cl. C12Q 1/6886; C12Q 1/44; C12Q 1/686

(52) U.S. Cl. CPC C12Q 1/6886; C12Q 1/44; C12Q 1/686; C12Q 2600/154

(57) ABSTRACT

Methods for assessment of subjects with hematuria and/or other suspicious urinary tract symptoms indicative of malignancy are provided, which are particularly useful for detecting primary bladder cancer or alternatively ruling-out bladder cancer (particularly high-grade bladder cancer) in these subjects. The methods provided are based on analysis of DNA methylation markers in DNA from cells of urine samples of the subjects.

### FIELD OF THE INVENTION

The present invention relates to assessment of subjects with hematuria and other suspicious urinary tract symptoms that may indicate malignancy, by analyzing DNA methylation markers in DNA from cells of urine samples of the subjects.

### BACKGROUND OF THE INVENTION

Hematuria and other lower urinary tract symptoms (LUTS) are common, estimated to account for over 20% of urology evaluations. Urologic etiologies for hematuria include malignancy, infection, inflammation, calculus disease, benign prostatic hyperplasia (BPH), and congenital or acquired anatomic abnormalities. Renal disease and anticoagulants treatment may also cause hematuria. Hematuria may also be confused with gynecological sources of bleeding, myoglobinuria, or pigmentation of the urine from the ingestion of certain foods and drugs. Hematuria can be defined as microhematuria (non-visible) and macrohematuria (bloody urine). Suspicion for microhematuria is often raised by a positive urine dipstick testing, and macrohematuria is often patient-reported. Any suspicion of hematuria must be confirmed by urine microscopy. Identification of 3 or more red blood cells in high-power field (RBC/HPF) are typically defined as hematuria, and the definition of micro vs. macrohematuria is set at the cut-off of 25 RBC/HPF.

According to AUA/SUFU guidelines (American Urological Association and Society of Urodynamics, Female Pelvic Medicine & Urogenital Reconstruction) for microhematuria published in 2020, multiple medical disciplines should be involved in the assessment of hematuria. Detection of a benign cause for hematuria, such as urinary tract infection or medical renal disease, do not rule-out the presence of an additional malignant etiology, and might still require urological attention and workup. Prevalence of bladder cancer was found to be 0.4% in the low-risk group, 1.0% in the intermediate risk group and 6.3% in the high-risk group.

Urological workup is adapted to the patient risk of malignancy, and includes cystoscopy, in which the bladder is examined visually using an endoscope (cystoscope), and imaging methods such as ultrasound and computed tomography (CT). The evaluation procedures are associated with various risks and patient discomfort, for example cystoscopy is invasive, unpleasant and associated with a risk of infection, and imaging procedures may involve exposure to radiation. From a healthcare system vantage point, the evaluation procedures are laborious, costly and require specially trained personnel. The high prevalence of hematuria that is not associated with cancer may lead to a large number of unnecessary evaluation procedures.

Urine cytology is recommended for the initial evaluation of patients with macrohematuria in combination with cystoscopy and CT, but not for those with microhematuria, and not as a standalone test that can rule-in or rule-out cancer. This is probably due to two aspects: first, its relatively high specificity, about 85-90% that would result in favorable PPV in the macrohematuria population, but in the microhematuria population a large number of individuals might still receive false-positive results, meaning that in many individuals the follow-up procedures are in effect unnecessary. Second, cytology's sensitivity is moderate (50-60%) for high-grade disease, so the test cannot reliably rule-out high-grade disease. Therefore, cytology is not recommended as a stand-alone test that can dictate the next step in patient evaluation, such as cystoscopy and imaging.

There is under-evaluation of hematuria and variations in care, with 23% at-risk patients reported to receive any type of imaging, and 13% undergoing cystoscopy. Women and African Americans are more prone to lower rates of hematuria evaluation. As a result, approximately 25% of bladder cancers are already diagnosed at an invasive stage (AUA/SUO NMIBC guidelines). Delays in diagnosis of bladder cancer have been suggested to contribute to a 34% increased risk of cancer-specific mortality and a 15% increased risk of all-cause mortality.

Thus, a bladder cancer biomarker with very high specificity—and accordingly a very low false-positive rate, that also has high sensitivity for high-grade cancers—that could preferably be assayed quickly and non-invasively in the community, at the time of the initial detection of hematuria, would be highly beneficial to identify patients who should be referred promptly to urological evaluation. Such a biomarker would be useful to reliably rule-out high-grade bladder cancer in subjects with hematuria and/or suspicious lower urinary tract symptoms, and reduce the number of unnecessary evaluation tests.

There is a need for improved methods for evaluating subjects with hematuria and other lower urinary tract symptoms, which can effectively identify patients at-risk of having bladder cancer (particularly high-grade bladder cancer) and referring them to urological evaluation prior to progression to an invasive disease.

### SUMMARY OF THE INVENTION

The present invention provides methods for assessment of subjects with hematuria and/or other lower urinary tract symptoms associated with suspicion of malignancy, which are particularly useful for detecting primary bladder cancer or alternatively ruling-out bladder cancer in these subjects. The invention is particularly useful in relation to high-grade bladder cancer.

The methods of the present invention are non-invasive and utilize a set of DNA methylation markers that can be detected in cells present in urine samples. More particularly, the methods of the present invention utilize a set of DNA methylation markers previously identified to be hypermethylated in bladder cancer DNA compared to normal DNA. It is now disclosed that these markers have exceptionally high specificity for bladder cancer in a specific population of subjects, namely subjects presenting with hematuria and/or additional lower urinary tract symptoms that are associated with suspicion of malignancy. As exemplified hereinbelow, the set of markers showed above 98% specificity. The set of markers was tested in two groups of subjects with suspicious lower urinary tract symptoms and without evidence of bladder cancer. Remarkably, the set of markers produced no (zero) false-positive results in one of the groups, and only three false-positive results in the other group, leading to 98%-100% specificity. In addition, the set of markers was tested in a group of subjects with a histologically-confirmed primary bladder cancer and was found to have particularly high sensitivity of 87.5% in detecting high-grade primary bladder cancer. Such a high specificity in subjects with hematuria and/or other suspicious lower urinary tract symptoms, along with high-grade sensitivity of the markers, is useful for identifying primary bladder cancer in this patient population or alternatively reliably ruling-out high-grade bladder cancer, and optimize the referral of hematuria patients for urological evaluation and timely cancer detection.

With the disclosed markers selected from a defined set of markers, high-grade bladder cancer can be detected or ruled out already at the initial evaluation of subjects with suspicious urinary tract symptoms, since the assay is non-invasive, reliable and simple to operate. The ability to detect or rule-out high-grade bladder cancer already at the initial evaluation stage is beneficial for both the timing of the evaluation (as soon as possible following detection of suspicious symptoms), and also the access to it for underserved populations, or under-evaluated populations, such as women and African Americans.

According to one aspect, the present invention provides a method for determining the presence or absence of primary bladder cancer in a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value; and
(c) detecting that the calculated score is above a pre-defined cutoff, thereby determining a positive likelihood for the presence of primary bladder cancer in the subject.

According to another aspect, the present invention provides a method for assessing a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is above a pre-defined cutoff, thereby determining a positive likelihood for the presence of bladder cancer in the subject; and
(d) mandating cystoscopy for the subject with the score above the pre-defined cutoff.

According to a further aspect, the present invention provides a method for ruling-out bladder cancer in a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value; and
(c) detecting that the calculated score is below a pre-defined cutoff, thereby ruling-out bladder cancer in the subject; and
(d) obviating cystoscopy in the subject.

According to a further aspect, the present invention provides a method for assessing a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is below a pre-defined cutoff, thereby ruling-out bladder cancer in the subject; and
(d) performing on the subject at least one assay selected from the group consisting of: serum creatinine measurement, glomerular filtration rate measurement, blood calcium measurement, blood uric acid measurement, urine pH measurement, urine calcium measurement, urine culture, urinary flow test, postvoid residual volume test, renal ultrasound, abdominal ultrasound and prostate ultrasound, to assess presence of a non-malignant genitourinary condition in the subject. Each assay represents a separate embodiment of the present invention.

In some embodiments, the non-malignant genitourinary condition is selected from the group consisting of urinary tract infection, renal infection, renal calculi, bladder calculi, benign prostatic hyperplasia (BPH), renal disease or injury, and congenital or acquired anatomic abnormalities.

According to a further aspect, the present invention provides a method for assessing a human subject with hematuria, the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is below a pre-defined cutoff; and
(d) performing on the subject repeated urinalysis within 6-12 months.

In some embodiments, the subject has one or more lower urinary tract symptoms (LUTS).

According to a further aspect, the present invention provides a method for assessing the presence or absence of high-grade primary bladder cancer in a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is above a pre-defined cutoff, thereby determining a positive likelihood for the presence of high-grade bladder cancer in the subject.

According to another aspect, the present invention provides a method for ruling-out high-grade primary bladder cancer in a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is below a pre-defined cutoff, thereby ruling-out high-grade bladder cancer in the subject; and
(d) obviating cystoscopy in the subject.

In some embodiments, the method further comprises performing on the subject assessment of the presence or absence of a non-malignant genitourinary condition.

In some embodiments, the assessment of the presence or absence of a non-malignant genitourinary condition comprises at least one assay selected from the group consisting of: serum creatinine measurement, glomerular filtration rate measurement, blood calcium measurement, blood uric acid measurement, urine pH measurement, urine calcium measurement, urine culture, urinary flow test, postvoid residual volume test, renal ultrasound, abdominal ultrasound and prostate ultrasound.

According to a further aspect, the present invention provides a method of managing a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is above a pre-defined cutoff; and
(d) performing confirmatory bladder cancer testing on the subject with the score above the pre-defined cutoff, comprising at least one of: standard cystoscopy, enhanced cystoscopy, urine cytology, physical examination, biopsy, imaging using at least one of computed tomography (CT), ultrasound and magnetic resonance imaging (MRI), and trans ureteral resection of bladder tumor (TURBT). Each possibility represents a separate embodiment of the present invention.

In some particular embodiments, the confirmatory bladder cancer testing is cystoscopy. Thus, in some embodiments, there is provided herein a method of managing a human subject with at least one symptom selected from hematuria and lower urinary tract symptoms (LUTS), the method comprising: (a) determining in DNA from cells of a urine sample of the subject a methylation value for at least one marker locus selected from the group consisting of SEQ ID NOs: 1-15; (b) calculating a score for the subject based on the methylation value; (c) detecting that the calculated score is above a pre-defined cutoff; and (d) performing cystoscopy on the subject with the score above the pre-defined cutoff.

In some embodiments, in which a positive or suspicious finding(s) is identified by the cystoscopy, the methods of managing the subject further comprise performing on the subject at least one of biopsy and resection of the finding(s) in surgery (TURBT). Each possibility represents a separate embodiment of the present invention. The specimen(s) are then examined by a pathologist to definitively confirm or rule-out the presence of a bladder tumor. Pathological diagnosis typically further includes characterizing the bladder cancer type, stage and grade. Patients with muscle invasive tumors might require further workup to assess the extent of tumor spread, including ultrasound (US), CT, PET-CT and MRI. Each possibility represents a separate embodiment of the present invention.

In some embodiments, the methods of managing a subject further comprises characterizing the bladder cancer type, stage and grade in a subject in which the presence of bladder cancer is confirmed.

In some embodiments, the methods of managing a subject further comprises administering treatment to a subject in which the presence of bladder cancer is confirmed and the type, stage and grade of the bladder cancer are characterized, to treat the bladder cancer. In some embodiments, the treatment comprises one or more of transurethral resection of bladder tumor (TURBT), cystectomy (partial or radical), lymphadenectomy, chemotherapy (intravesical or systemic), radiation therapy, immunotherapy (intravesical or systemic) and targeted therapy. Intravesical chemotherapy may include one or more of mitomycin, gemcitabine, epirubicin and valrubicin. Intravesical chemotherapy may be given as is, as hyperthermic intravesical chemotherapy, as microwave-induced hyperthermia or as electromotive drug administration. Systemic immunotherapy may include one or more of platinum-based (for example, cisplatin, carboplatin) fluorouracil (5-FU), mitomycin, gemcitabine, methotrexate, vinblastine, doxorubicin, docetaxel, paclitaxel and vinflunine. Intravesical immunotherapy may include one or more of Bacillus Calmette-Guerin (BCG), interferon-α and Nadofaragene firadenovec. Systemic immunotherapy may include one or more of immune checkpoint inhibitors, such as PD-1 and PD-L1 inhibitors (for example: avelumab, nivolumab, atezolizumab and pembrolizumab) and antibody-drug conjugates, such as enfortumab vedotin and sacituzumab govitecan. Targeted therapy may include fibroblast growth factor receptor (FGFR) inhibitors such as erdafitinib. Treatment may further include supportive care. Various treatment options may be combined, (e.g., chemoradiation, instillation combination therapy). Each treatment possibility and combination represents a separate embodiment of the present invention.

The type of treatment is determined by skilled practitioner(s) according to characteristics of the tumor, including the type, stage and grade of the tumor. The type of treatment is determined typically also based on additional factors such as characteristics of the patient.

According to another aspect, there is provided herein a method for managing a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is below a pre-defined cutoff, thereby ruling-out bladder cancer in the subject and obviating cystoscopy; and
(d) performing on the subject at least one assay selected from the group consisting of: serum creatinine measurement, glomerular filtration rate measurement, blood calcium measurement, blood uric acid measurement, urine pH measurement, urine calcium measurement, urine culture, urinary flow test, postvoid residual volume test, renal ultrasound, abdominal ultrasound and prostate ultrasound, to assess the presence of a non-malignant genitourinary condition.

According to a further aspect, there is provided herein a method for managing a human subject with hematuria, the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is below a pre-defined cutoff; and
(d) performing on the subject urinalysis within 6-12 months.

In some embodiments, the lower urinary tract symptoms (LUTS) comprise at least one symptom selected from: increased urinary frequency, urinary urgency, urge incontinence, nocturia, changes in urine stream, urinary hesitating, straining and dribbling, and incomplete emptying. Each possibility represents a separate embodiment of the present invention.

In some embodiments, the hematuria is microhematuria. In other embodiments, the hematuria is macrohematuria.

In some embodiments, the at least one marker locus comprises one or more (and preferably a plurality of) marker loci selected from the group consisting of SEQ ID NOs: 1-15.

In some embodiments, the at least one marker locus comprises the locus set forth in SEQ ID NO: 1. In some embodiments, the at least one marker locus further comprises the locus set forth in SEQ ID NO: 5. In some embodiments, the at least one marker locus comprises the loci set forth in SEQ ID NO: 1, SEQ ID NO: 5, SEQ ID NO: 7 and SEQ ID NO: 11. In some embodiments, the at least one marker locus comprises the loci set forth in SEQ ID NO: 1, SEQ ID NO: 5, SEQ ID NO: 7 and SEQ ID NO: 11, and further comprises at least one additional marker locus selected from the group of loci set forth in SEQ ID NO: 2, SEQ ID NO: 3, SEQ ID NO: 4, SEQ ID NO: 6, SEQ ID NO: 8, SEQ ID NO: 9, SEQ ID NO: 10, SEQ ID NO: 12, SEQ ID NO: 13, SEQ ID NO: 14 and SEQ ID NO: 15. In additional embodiments, the at least one additional marker locus comprises the loci set forth in SEQ ID NO: 2, SEQ ID NO: 3, SEQ ID NO: 4, SEQ ID NO: 6, SEQ ID NO: 8, SEQ ID NO: 9, SEQ ID NO: 10, SEQ ID NO: 12, SEQ ID NO: 13, SEQ ID NO: 14 and SEQ ID NO: 15.

In some embodiments, the methylation value is determined using one or more analysis methods selected from the group consisting of DNA sequencing, real-time PCR, and array hybridization.

In some embodiments, determining the methylation value comprises, for a plurality of potential methylation sites in a plurality of marker loci, quantifying methylated read counts at the potential methylation sites and calculating a methylation value based on the number of methylated read counts.

In some embodiments, determining a methylation value for at least one marker locus selected from the group consisting of SEQ ID NOs: 1-15 and calculating a score comprise: (i) subjecting the DNA from cells of the urine sample of the subject to digestion with at least one methylation-sensitive restriction endonuclease recognizing a sequence within the at least one marker locus that is hypermethylated in cancer DNA compared to non-cancer DNA, to obtain restriction endonuclease-treated DNA; (ii) co-amplifying from the restriction endonuclease-treated DNA the at least one marker locus and a control locus, thereby generating an amplification product for each locus; (iii) determining a signal intensity for each generated amplification product; and (iv) comparing a ratio between the signal intensities of the amplification products of each of said at least one marker locus and the control locus to at least one reference ratio selected from cancer reference ratio and non-cancer reference ratio, to calculate a score.

In some embodiments, step (i) is performed using a single methylation-sensitive restriction endonuclease. In some embodiments, the methylation-sensitive restriction endonuclease is HhaI. In additional embodiments, the methylation-sensitive restriction endonuclease is HinP1I.

In some embodiments, the control locus is a locus that does not contain a nucleotide sequence recognized by the methylation-sensitive restriction endonuclease.

In some embodiments, the methylation-sensitive restriction endonuclease is selected from HhaI and HinP1I, and the control locus is the control locus is the locus set forth in SEQ ID NO: 16.

In some embodiments, step (ii) is performed using real-time PCR. In some embodiments, step (ii) comprises adding fluorescent probes for assisting in detecting the amplification products of the at least one marker locus and the control locus. In some embodiments, determining the methylation value comprises, for a plurality of marker loci, determining a ΔCq for each marker locus and calculating a single composite methylation value based on the ΔCq determined for each marker locus. In some embodiments, the ratio between the signal intensities of the amplification products of each of said at least one marker locus and the control locus is calculated by determining the quantification cycle (Cq) for each locus and calculating 2^{(Cq control locus-Cq restriction locus)}.

These and further aspects and features of the present invention will become apparent from the detailed description, examples and claims which follow.

### DETAILED DESCRIPTION OF THE INVENTION

The present invention relates to assessment of subjects with hematuria and other suspicious lower urinary tract symptoms (LUTS) that may indicate malignancy, by analyzing DNA methylation markers in DNA from cells of urine samples of the subjects.

Advantageously, the DNA methylation markers are suitable for methylation analysis using enzymatic digestion of DNA with at least one methylation-sensitive restriction enzyme followed by real-time PCR of the methylation markers and an internal reference locus, and subsequently precise quantification of ratios between the signals obtained from each marker locus and the signal of the internal reference locus. Thus, in some embodiments, methylation analysis according to the present invention does not require evaluating absolute methylation levels at the analyzed genomic loci, but rather calculating a signal ratio (reflecting the methylation ratio) between the analyzed genomic loci and an internal reference locus in the same sample. This is in contrast to conventional methods utilizing methylation analysis for distinguishing between tumor-derived and normal DNA, which require determining actual methylation levels at specific genomic loci. Thus, embodiments of the present invention eliminate the need for standard curves and/or additional laborious steps involved in determination of methylation levels per se, thereby offering a simple and cost-effective procedure. An additional advantage over known approaches for analyzing methylation is conferred by the signal ratios obtained according to some embodiments of the present invention, which are calculated between loci amplified in the same reaction mixture (i.e., under the same reaction conditions). This renders the methods insensitive to various “noise” factors, such as changes in template DNA concentration, PCR conditions, and presence of inhibitors. Such noises are inherent for methods that are based on quantifying methylation levels of loci by comparing signals from separate amplification reactions.

Methylation in the human genome occurs in the form of 5-methyl cytosine and is confined to cytosine residues that are part of the sequence CG, also denoted as CpG dinucleotides (cytosine residues that are part of other sequences are not methylated). Some CpG dinucleotides in the human genome are methylated, and others are not. In addition, methylation is cell and tissue specific, such that a specific CpG dinucleotide can be methylated in a certain cell and at the same time unmethylated in a different cell, or methylated in a certain tissue and at the same time unmethylated in different tissues. DNA methylation is an important regulator of gene transcription. Aberrant DNA methylation patterns, both hypermethylation and hypomethylation compared to normal tissue, have been associated with a large number of human malignancies.

The term “bladder cancer” refers to a cancer that occurs in the urinary bladder and includes transitional cell carcinoma (TCC, also referred to as urothelial cell carcinoma), squamous cell carcinoma, adenocarcinoma, small cell carcinoma and sarcoma. Each possibility represents a separate embodiment of the present invention. As used herein, “bladder cancer” does not include cancers that occur at other parts of the urinary systems, such as upper tract urothelial carcinoma (UTUC).

Most bladder cancers are transitional cell carcinomas, arising from epithelial cells in the inner lining of the bladder (urothelium). Other types of bladder cancers include squamous cell carcinomas, adenocarcinomas, sarcomas and small cell carcinomas.

TCC, arising from epithelial cells in the inner lining of the bladder (urothelium), is the most common type of bladder cancer accounting for more than 90% of the cases. TCC typically includes two sub-types, papillary carcinomas, in which the tumors grow in slender, finger-like projections from the inner surface of the bladder toward the hollow center, and flat carcinomas, in which tumors do not grow toward the hollow part of the bladder. Squamous cell carcinoma and adenocarcinoma are less common types of bladder cancer, and small cell carcinoma and sarcoma are relatively rare.

Bladder cancer may be typically further classified as either non-invasive, where the cancer cells are confined to the inner layer of the transitional epithelium, or invasive, where cancer cells grow into the lamina propria of the bladder or even deeper into the muscle layer. A bladder cancer can also be described as superficial or non-muscle invasive. These terms include both non-invasive tumors as well as any invasive tumors that have not grown into the main muscle layer of the bladder.

One of the common classification of bladder cancer is the T category which describes how far the main tumor has grown into the wall of the bladder (or beyond). Typically, bladder cancers start in the urothelium and, as the cancer grows, may invade into other layers in the bladder, thus becoming more advanced. Classification according to the T category includes the following terms:
T0: No evidence of a primary tumor
Ta: Non-invasive papillary carcinoma
Tis or CIS: Non-invasive flat carcinoma (carcinoma in situ)
T1: The tumor has grown from the layer of cells lining the bladder into the connective tissue below. It has not grown into the muscle layer of the bladder.
T2: The tumor has grown into the muscle layer.
   T2a: The tumor has grown only into the inner half of the muscle layer.
   T2b: The tumor has grown into the outer half of the muscle layer.
T3: The tumor has grown through the muscle layer of the bladder and into the fatty tissue layer that surrounds it.
   T3a: The spread to fatty tissue can only be seen by using a microscope.
   T3b: The spread to fatty tissue is large enough to be seen on imaging tests or to be seen or felt by the surgeon.
T4: The tumor has spread beyond the fatty tissue and into nearby organs or structures. It may be growing into any of the following: the stroma (main tissue) of the prostate, the seminal vesicles, uterus, vagina, pelvic wall, or abdominal wall.
   T4a: The tumor has spread to the stroma of the prostate (in men), or to the uterus and/or vagina (in women).
   T4b: The tumor has spread to the pelvic wall or the abdominal wall.

It is also common to describe bladder cancer by its grade (G), which represents the similarity of cancer cells to healthy cells when viewed under a microscope. Healthy tissue usually contains various types of cells grouped together. If the cancer looks similar to healthy tissue and contains different cell groupings, it is called differentiated or a low-grade tumor. If the cancerous tissue looks very different from healthy tissue, it is called poorly differentiated or a high-grade tumor.

Urologic surgeons may also classify a tumor's grade based on its chance to recur or progress (grow and spread) in order to plan treatment based on the grade, using the following categories:
Papilloma (or benign papillary urothelial neoplasm of low malignant potential; PUNLMP)—may recur but has a low risk of progressing.
Low grade: likely to recur and progress compared with PUNLMP.
High grade: most likely to recur and progress.

Bladder cancer can sometimes affect many areas of the bladder at the same time. If more than one tumor is found, the letter m is added to the appropriate T category. If the cancer is advanced, lymph nodes involvement and metastases typically are also assessed to complete full staging based on Tumor, Node, Metastasis (TNM) staging system, in which T describes the size of the tumor and any spread of cancer into nearby tissue; N describes spread of the cancer to nearby lymph nodes; and M describes metastasis.

The term “subject” as used herein is interchangeable with “individual” and refers to a human subject. The subject has hematuria and/or at least one lower urological symptom that may indicate malignancy of the bladder, including at least one of: increased urinary frequency, urinary urgency, urge incontinence, nocturia, changes in urine stream, urinary hesitating, straining and dribbling, and incomplete emptying. The hematuria may be microhematuria, defined as ≥3 red blood cells observed per high-power field (RBC/HPF) on microscopic evaluation of a single, properly collected urine specimen, or macrohematuria (also termed gross hematuria), where there is visible blood in the urine. Macrohematuria is also defined as ≥25 RBC/HPF. Each possibility represents a separate embodiment of the present invention.

The subject may be suspected of having bladder cancer. In some embodiments, the subject may be at risk of developing bladder cancer, for example, based on genetic predisposition, and/or family history. A subject evaluated using the methods of the present invention is a subject without a history of bladder cancer or other urothelial cancer (the subject was not previously diagnosed with bladder cancer or upper tract urothelial carcinoma).

### Urine Sample Collection and Processing

The DNA to be analyzed by the methods and systems of the present invention is cellular DNA obtained from cells present in the urine. The present invention can be carried out by processing a urine sample or cells which were previously collected from a urine sample. In some embodiments, a urine sample is processed to collect the cells, after which the DNA is extracted from the collected cells. Collection of the cells may be carried out using centrifugation or by the use of other methods that capture the cells, for example, a filtration device which collects the cells without needing centrifugation, such as the filtration device described in Andersson et al., 2015, PLOS ONE, 10 (7): e0131889).

In some embodiments, urine samples may be collected from subjects using conventional collection containers or tubes. Preferably, at least 10 ml of urine are collected.

In some embodiments, methods of the invention may include a step of extracting genomic DNA from a urine sample. This can be achieved according to methods known in the art. Exemplary procedures are described, e.g., in Sambrook et al, Molecular Cloning: A Laboratory Manual, Fourth Ed., Cold Spring Harbor Laboratory Press, Cold Spring Harbor, NY, 2012.

In some embodiments, a urine sample to be analyzed is subjected to centrifugation to create a cell pellet, and DNA is then extracted from the cell pellet. This can be achieved using a suitable DNA extraction buffer, for example as described in the Examples section hereinbelow.

Preferably, the DNA sample on which the methylation analysis is carried out is substantially free of single-stranded DNA (ssDNA). As used herein, “substantially free of ssDNA” or “substantially devoid of ssDNA” indicates a DNA sample in which less than 7% of the DNA is ssDNA, preferably less than 5% of the DNA is ssDNA, more preferably less than 1% of the DNA is ssDNA (namely, at least 99% of the DNA is double-stranded) (by number of molecules). In some embodiments, the DNA sample contains less than 0.1% ssDNA. In some embodiments, the DNA sample contains less than 0.01% ssDNA. In some embodiments, the DNA sample contains no ssDNA (free of ssDNA). Extraction of DNA to obtain a DNA sample substantially free of ssDNA is described, for example, in WO 2020/188561, assigned to the Applicant of the present invention.

In some embodiments, all DNA that was extracted is used according to the present invention. In some embodiments, the DNA is not quantified prior to the methylation analysis according to the present invention.

Methods of the invention may be performed on a urine sample, from which DNA is then extracted, or may be performed on cells which have already been obtained from a urine sample (from which DNA will then be extracted), or may be performed on DNA which has already been extracted from a urine sample or from cells therein.

### Methylation Value

A “methylation value” as used herein is a numerical value representing the level of methylation of a particular genomic locus in a DNA sample. As methylation may be analyzed and measured using various methods, a “methylation value” according to the present invention may be expressed as a variety of numerical values. For example, a methylation value may be a methylation level expressed as a ratio or percentage of the DNA molecules that are methylated at a marker locus out of the total number of DNA molecules containing the marker locus in the sample. As a further example, a methylation value may be a methylation level expressed as a copy number of methylated DNA molecules at the marker locus (e.g., read count obtained following sequencing, as will be described in more detail below). As yet a further example, a methylation value may be a methylation level expressed as an intensity of a signal obtained from a marker locus, e.g., fluorescent signal obtained using a detectable fluorescent label/probe. A methylation value may be normalized with respect to a reference locus and/or a reference DNA sample. In some particular embodiments, the methylation value is a methylation ratio between a marker locus and a control locus, expressed as a ratio between signals obtained for these loci following methylation-sensitive enzymatic digestion of the DNA sample and PCR amplification, as will be described in more detail below.

In some embodiments, the methods of the present invention comprise determining a methylation value for the marker locus set forth in SEQ ID NO: 1, and optionally for at least one additional marker locus. In some embodiments, the methods of the present invention comprise determining a methylation value for the marker locus set forth in SEQ ID NO: 2, and optionally for at least one additional marker locus. In some embodiments, the methods of the present invention comprise determining a methylation value for the marker locus set forth in SEQ ID NO: 3, and optionally for at least one additional marker locus. In some embodiments, the methods of the present invention comprise determining a methylation value for the marker locus set forth in SEQ ID NO: 4, and optionally for at least one additional marker locus. In some embodiments, the methods of the present invention comprise determining a methylation value for the marker locus set forth in SEQ ID NO: 5, and optionally for at least one additional marker locus. In some embodiments, the methods of the present invention comprise determining a methylation value for the marker locus set forth in SEQ ID NO: 6, and optionally for at least one additional marker locus. In some embodiments, the methods of the present invention comprise determining a methylation value for the marker locus set forth in SEQ ID NO: 7, and optionally for at least one additional marker locus. In some embodiments, the methods of the present invention comprise determining a methylation value for the marker locus set forth in SEQ ID NO: 8, and optionally for at least one additional marker locus. In some embodiments, the methods of the present invention comprise determining a methylation value for the marker locus set forth in SEQ ID NO: 9, and optionally for at least one additional marker locus. In some embodiments, the methods of the present invention comprise determining a methylation value for the marker locus set forth in SEQ ID NO: 10, and optionally for at least one additional marker locus. In some embodiments, the methods of the present invention comprise determining a methylation value for the marker locus set forth in SEQ ID NO: 11, and optionally for at least one additional marker locus. In some embodiments, the methods of the present invention comprise determining a methylation value for the marker locus set forth in SEQ ID NO: 12, and optionally for at least one additional marker locus. In some embodiments, the methods of the present invention comprise determining a methylation value for the marker locus set forth in SEQ ID NO: 13, and optionally for at least one additional marker locus. In some embodiments, the methods of the present invention comprise determining a methylation value for the marker locus set forth in SEQ ID NO: 14, and optionally for at least one additional marker locus. In some embodiments, the methods of the present invention comprise determining a methylation value for the marker locus set forth in SEQ ID NO: 15, and optionally for at least one additional marker locus.

As described herein, each marker locus of the present invention contains a plurality of differentially methylated CG dinucleotides located within restriction sites of methylation-sensitive restriction endonucleases. In some embodiments, when the marker loci are analyzed using methylation-sensitive enzymatic digestion of the DNA, the methylation value for a marker locus is based on CG dinucleotides within restriction site(s) of the methylation-sensitive restriction endonuclease(s) used in the assay.

The following sections describe methylation analyses according to some embodiments of the present invention, which are based on methylation-sensitive enzymatic digestion of the DNA sample followed by quantitative PCR amplification and analysis of amplification products, or methylation-sensitive enzymatic digestion of the DNA sample followed by high-throughput sequencing (next-generation sequencing). A person of skill in the art would appreciate that other methods for analyzing methylation and obtaining methylation value(s) of marker loci may be used with the present invention.

### Methylation Analysis Using Methylation-Sensitive Enzymatic Digestion and PCR Amplification

### A. DNA Digestion

In some embodiments, following extraction the DNA is subjected to digestion with at least one methylation-sensitive restriction endonuclease, for example, with one, two, three methylation-sensitive restriction endonucleases. Each number of endonucleases used in the assay represents a separate embodiment of the present invention.

In some embodiments, the entire DNA that is extracted from the urine sample is used in the digestion step. In some embodiments, the DNA is not quantified prior to being subjected to digestion. In other embodiments, the DNA may be quantified prior to digestion thereof.

A “restriction endonuclease”, used herein interchangeably with a “restriction enzyme”, refers to an enzyme that cuts DNA at or near specific recognition nucleotide sequences, known as restriction sites. Restriction sites are usually 4 to 8 nucleotide long and are typically palindromic (i.e., reading in a certain direction, e.g. 5′ to 3′, on one strand is identical to the sequence in the same direction (5′ to 3′) on the complementary strand).

A “methylation-sensitive” restriction endonuclease is a restriction endonuclease that cleaves its recognition sequence only if it is unmethylated (while methylated sites remain intact). Thus, the extent of digestion of a DNA sample by a methylation-sensitive restriction endonuclease depends on the methylation level, where a higher methylation level protects from cleavage and accordingly results in less digestion.

Examples of methylation-sensitive restriction endonucleases that may be used according to the present invention include: AciI, AfeI, ApaI, AscI, AvaI, AvaII, BanI, BbeI, BcgI, BfuCI, BsaAI, BsaHI, BsaI, BseYI, BsiEI, BsII, BsmAI, BsmFI, BsrBI, BsrFI, BssHII, BssKI, BstUI, Cac8I, DpnI, EciI, FauI, Fnu4HI, FspI, HaeII, HgaI, HhaI, HinfI, HinPII, HpaII, HpyI66ii, HpyI88iii, Hpy99I, HpyCH4IV, KasI, MmeI, MspAII, MwoI, NheI, NlaIV, PleI, PmlI, PspOMI, SacII, Sau3AI, Sau96I, ScrFI, SfoI, SgrAI, SmaI, TfiI, TscI, Tsel, and TspMI, and their ‘HF’ high fidelity variants where available. Each possibility represents a separate embodiment of the present invention.

In general, embodiments which can be performed with methylation-sensitive restriction endonuclease(s) can be done alternatively with methylation-dependent restriction endonucleases, and downstream steps will be adjusted accordingly.

In some embodiments, the DNA extracted from the urine sample may be subjected to digestion with a single methylation-sensitive restriction endonuclease. In some particular embodiments, the methylation-sensitive restriction endonuclease may be HhaI. In additional particular embodiments, the methylation-sensitive restriction endonuclease may be HinP1I. In other embodiments, the DNA extracted from the urine sample may be subjected to digestion with a plurality of methylation-sensitive restriction endonucleases. As used herein, “a plurality” indicates “at least two”.

In some embodiments, DNA digestion may be carried out to complete digestion. In some embodiments, the methylation-sensitive restriction endonuclease may be HhaI, and complete digestion may be achieved following one to two hours incubation with the enzyme at 37° C.

In some embodiments, DNA digestion may be carried out to complete digestion. In some embodiments, the methylation-sensitive restriction endonuclease may be HinP1I, and complete digestion may be achieved following one to two hours incubation with the enzyme at 37° C.

### B. Amplification of Genomic Loci

The terms “genomic locus” or “locus” as used herein are interchangeable and refer to a DNA sequence at a specific position on a chromosome. The specific position may be identified by the molecular location, namely, by the numbers of the starting and ending base pairs on the chromosome. A variant of a DNA sequence at a given genomic position is called an allele. Alleles of a locus are located at identical sites on homologous chromosomes. Loci include gene sequences as well as other genetic elements (e.g., intergenic sequences).

A “marker locus” as disclosed herein refers to a genomic locus that is differentially methylated between sources of DNA, and therefore analysis of its methylation provides an indication with respect to the source of the DNA.

A “control locus” and “internal reference locus” are interchangeable and used herein to describe a locus the digestion of which, with the restriction enzyme applied in the digestion step, is independent of the presence or absence of methylation. In some embodiments, the control locus is a locus that exhibits the same digestion and amplification profile in bladder cancer and in healthy tissue. In some embodiments, the control locus is a locus devoid of the recognition sequence of the restriction enzyme applied in the digestion step, and the sequence of the control locus remains intact regardless of its methylation status when the DNA sample is digested. Advantageously, the control locus is an internal locus, i.e. a locus within the analyzed DNA sample, thus eliminating the need for external/additional control sample(s).

A marker locus for use according to the present invention include any one or more of the loci set forth in SEQ ID NOs: 1-15, as follows (numbering according to the hg18 build of the human genome):
SEQ ID NO: 1, corresponds to position 65676359-65676418 on chromosome 17, (KCNJ2 gene);
SEQ ID NO: 2, corresponds to position 21958446-21958585 on chromosome 9, (CDKN2A gene);
SEQ ID NO: 3, corresponds to position 336844-336903 on chromosome 6, (IRF4 gene);
SEQ ID NO: 4, corresponds to position 33319507-33319636 on chromosome 21, (Olig2 gene);
SEQ ID NO: 5, corresponds to position 166502151-166502220 on chromosome 6, intergenic region;
SEQ ID NO: 6, corresponds to position 896902-897031 on chromosome 18, (ADCYAP1 gene);
SEQ ID NO: 7, corresponds to position 32747873-32748022 on chromosome 5, (NPR3 gene);
SEQ ID NO: 8, corresponds to position 27949195-27949264 on chromosome 6, intergenic region;
SEQ ID NO: 9, corresponds to position 27191603-27191672 on chromosome 7, (HOXA9 gene);
SEQ ID NO: 10, corresponds to position 170170302-170170361 on chromosome 16, intergenic region;
SEQ ID NO: 11, corresponds to position 30797737-30797876 on chromosome 15, intergenic region;
SEQ ID NO: 12, corresponds to position 7936767-7936866 on chromosome 1, intergenic region;
SEQ ID NO: 13, corresponds to position 170077565-170077634 on chromosome 1, (DNM3 gene);
SEQ ID NO: 14, corresponds to position 1727592-1727661 on chromosome 2, (PXDN gene); and
SEQ ID NO: 15, corresponds to position 72919092-72919231 on chromosome 8, (MSC gene).

The marker loci set forth in SEQ ID NOs: 1-15, previously disclosed in WO 2017/006317, assigned to the Applicant of the present invention, are differentially methylated between cancerous bladder tissue and normal bladder tissue. More particularly, these loci have increased methylation in bladder cancer tissue compared to normal tissue.

Each of these loci contains CG dinucleotides that are more methylated in DNA from cancerous bladder tissue compared to DNA from normal non-cancerous bladder tissue. Advantageously, the differentially methylated CG dinucleotides are located within recognition sites of methylation-sensitive restriction enzymes.

In some embodiments, each of these loci may contain at least one restriction site of a methylation-sensitive restriction enzyme in which the CG dinucleotide is more methylated in bladder cancer cells than in normal cells, meaning that in the cancerous tissue a greater number of cells contain methylation at this position compared to normal tissue. In some embodiments, each of these loci may contain at least one HhaI restriction site (GCGC). In some embodiments, each of these loci may contain at least one HinP1I restriction site. The methylation-sensitive restriction enzyme cleaves its recognition sequence only if it is unmethylated. Thus, a DNA sample containing a higher percentage of DNA molecules in which the CG dinucleotide in the restriction site is methylated would be digested to a lesser extent compared to a DNA sample containing a higher percentage of DNA molecules in which the CG dinucleotide is unmethylated. Based on the methods disclosed herein, DNA digestion by methylation-sensitive restriction enzymes is less extensive for DNA from urine samples of bladder cancer patients compared to DNA from normal (healthy) individuals. The difference in digestion efficiency establishes different amplification patterns in subsequent amplification and quantification steps, which enables distinguishing between DNA from a cancerous bladder tissue and DNA from a normal bladder tissue.

In some embodiments, each of the loci set forth in SEQ ID NOs: 1-15 may contain additional CG dinucleotides whose methylation status is of no relevance or influence on the assay-only methylation at the recognition sequence of the restriction enzyme (e.g. HhaI or HinP1I) is relevant.

In some embodiments, the control locus is as set forth in SEQ ID NO: 16, which corresponds to position 121380854-121380913 on chromosome 7 (intergenic region). In some embodiments, the control locus, also termed an internal reference locus, does not contain a recognition sequence of the restriction enzyme. In some embodiment, the sequence of the control locus remains intact (regardless of its methylation status) when a DNA sample is digested with a methylation-sensitive restriction enzyme.

In some embodiments, the sequence of the control locus exhibits the same digestion and amplification profile in bladder cancer tissue and in a healthy bladder tissue.

In some embodiments, the control locus comprises the locus set forth in SEQ ID NO: 16 and the amplification pattern of the control locus following digestion with the methylation sensitive restriction enzyme is not affected by methylation.

In some embodiments, the methods of the present invention comprise amplifying at least one marker locus and at least one control locus following digestion of the DNA sample.

As used herein, “at least one (marker/control) locus”, may encompass a single locus or a plurality of separate loci.

In some embodiments, the methods of the present invention comprise amplifying the marker locus set forth in SEQ ID NO: 1 and a control locus, and optionally at least one additional marker locus. In some embodiments, the methods of the present invention comprise amplifying the marker locus set forth in SEQ ID NO: 2 and a control locus, and optionally at least one additional marker locus. In some embodiments, the methods of the present invention comprise amplifying the marker locus set forth in SEQ ID NO: 3 and a control locus, and optionally at least one additional marker locus. In some embodiments, the methods of the present invention comprise amplifying the marker locus set forth in SEQ ID NO: 4 and a control locus, and optionally at least one additional marker locus. In some embodiments, the methods of the present invention comprise amplifying the marker locus set forth in SEQ ID NO: 5 and a control locus, and optionally at least one additional marker locus. In some embodiments, the methods of the present invention comprise amplifying the marker locus set forth in SEQ ID NO: 6 and a control locus, and optionally at least one additional marker locus. In some embodiments, the methods of the present invention comprise amplifying the marker locus set forth in SEQ ID NO: 7 and a control locus, and optionally at least one additional marker locus. In some embodiments, the methods of the present invention comprise amplifying the marker locus set forth in SEQ ID NO: 8 and a control locus, and optionally at least one additional marker locus. In some embodiments, the methods of the present invention comprise amplifying the marker locus set forth in SEQ ID NO: 9 and a control locus, and optionally at least one additional marker locus. In some embodiments, the methods of the present invention comprise amplifying the marker locus set forth in SEQ ID NO: 10 and a control locus, and optionally at least one additional marker locus. In some embodiments, the methods of the present invention comprise amplifying the marker locus set forth in SEQ ID NO: 11 and a control locus, and optionally at least one additional marker locus. In some embodiments, the methods of the present invention comprise amplifying the marker locus set forth in SEQ ID NO: 12 and a control locus, and optionally at least one additional marker locus. In some embodiments, the methods of the present invention comprise amplifying the marker locus set forth in SEQ ID NO: 13 and a control locus, and optionally at least one additional marker locus. In some embodiments, the methods of the present invention comprise amplifying the marker locus set forth in SEQ ID NO: 14 and a control locus, and optionally at least one additional marker locus. In some embodiments, the methods of the present invention comprise amplifying the marker locus set forth in SEQ ID NO: 15 and a control locus, and optionally at least one additional marker locus.

In some embodiments, the methods of the present invention comprise amplifying a marker locus selected from SEQ ID NOs: 1-15, and a control locus as set forth in SEQ ID NO: 16.

In some embodiments, the methods of the present invention comprise amplifying a plurality of marker loci (i.e., at least two marker loci) and a control locus.

In some embodiments, the plurality of marker loci comprises the locus set forth in SEQ ID NO: 1 and the locus set forth in SEQ ID NO: 5. In some embodiments, the plurality of marker loci further comprises at least one marker locus selected from the loci set forth in SEQ ID NO: 7 and SEQ ID NO: 11. In some embodiments, the plurality of marker loci comprises the loci set forth in SEQ ID NO: 1, SEQ ID NO: 5, SEQ ID NO: 7 and SEQ ID NO: 11.

In some embodiments, the plurality of marker loci comprises the loci set forth in SEQ ID NO: 1, SEQ ID NO: 5, SEQ ID NO: 7 and SEQ ID NO: 11, and further comprises at least one additional marker locus selected from the group consisting of SEQ ID NO: 2, SEQ ID NO: 3, SEQ ID NO: 4, SEQ ID NO: 6, SEQ ID NO: 8, SEQ ID NO: 9, SEQ ID NO: 10, SEQ ID NO: 12, SEQ ID NO: 13, SEQ ID NO: 14 and SEQ ID NO: 15. Each possibility represents a separate embodiment of the present invention.

In some embodiments, the plurality of marker loci comprises the locus set forth as SEQ ID NO: 1 and further comprises one, two, three, four, five, six, seven, eight, nine, ten, eleven, twelve, thirteen or fourteen additional marker loci selected from the group consisting of SEQ ID NOs: 2-15. Each possibility represents a separate embodiment of the present invention.

In some embodiments, the plurality of marker loci comprises the loci set forth as SEQ ID NOs: 1 and 5, and further comprises one, two, three, four, five, six, seven, eight, nine, ten, eleven, twelve or thirteen additional marker loci selected from the group consisting of SEQ ID NO: 2, SEQ ID NO: 3, SEQ ID NO: 4, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15. Each possibility represents a separate embodiment of the present invention.

In some embodiments, the plurality of marker loci comprises the loci set forth as SEQ ID NOs: 1, 5 and 7, and further comprises one, two, three, four, five, six, seven, eight, nine, ten, eleven or twelve additional marker loci selected from the group consisting of SEQ ID NO: 2, SEQ ID NO: 3, SEQ ID NO: 4, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15. Each possibility represents a separate embodiment of the present invention. In some embodiments, the plurality of marker loci comprises the loci set forth as SEQ ID NOs: 1, 5, 7 and 11, and further comprises one, two, three, four, five, six, seven, eight, nine, ten or eleven additional marker loci selected from the group consisting of SEQ ID NO: 2, SEQ ID NO: 3, SEQ ID NO: 4, SEQ ID NO: 6, SEQ ID NO: 8, SEQ ID NO: 9, SEQ ID NO: 10, SEQ ID NO: 12, SEQ ID NO: 13, SEQ ID NO: 14 and SEQ ID NO: 15. Each possibility represents a separate embodiment of the present invention.

In some embodiments, the plurality of marker loci comprises the loci set forth in SEQ ID NOs: 1-15. In some embodiments, the plurality of marker loci is consisting of the loci set forth in SEQ ID NOs: 1-15. In some embodiments, the methods of the present invention comprise amplifying a plurality of marker loci as set forth SEQ ID NOs: 1-15.

As used herein, “amplification” refers to an increase in the number of copies of one or more particular nucleic acid target of interest. Amplification is typically performed by polymerase chain reaction (PCR) in the presence of a PCR reaction mixture which may include a suitable buffer supplemented with the DNA template, polymerase (usually Taq Polymerase), dNTPs, primers and probes (as appropriate).

The term “polynucleotide” as used herein include polymeric forms of nucleotides of any length, either deoxyribonucleotides or ribonucleotides, or analogs thereof. The term “oligonucleotide” is also used herein to include a polymeric form of nucleotides, typically of up to 100 bases in length.

An “amplification product” collectively refers to nucleic acid molecules of a particular target sequence that are generated and accumulated in an amplification reaction. The term generally refers to nucleic acid molecules generated by PCR using a given set of amplification primers.

As used herein, a “primer” defines an oligonucleotide which is capable of annealing to (hybridizing with) a target sequence, thereby creating a double stranded region which can serve as an initiation point for DNA synthesis under suitable conditions. The terminology “primer pair” refers herein to a pair of oligonucleotides which are selected to be used together in amplifying a selected nucleic acid sequence by one of a number of types of amplification processes, preferably PCR. As commonly known in the art, the primers may be designed to bind to a complementary sequence under selected conditions.

The primers may be of any suitable length, depending on the particular assay format and the particular needs. In some embodiments, the primers may include at least 15 nucleotides in length, preferably between 15-25 nucleotides in length, or 19-25 nucleotides in length. The primers may be adapted to be especially suited to a chosen nucleic acid amplification system. The oligonucleotide primers may be designed by taking into consideration the melting point of hybridization thereof with their targeted sequence (Sambrook et al, ibid).

In some embodiments, the marker and control loci may be amplified from the same DNA sample (the digested sample) using pairs of reverse and forward primers designed to specifically amplify each locus. In some embodiments, the primers may be designed to amplify a locus along with 5′ and 3′ flanking sequences thereof.

In some embodiments, the 5′ flanking sequences may include between 1-60 bases immediately upstream of the locus. In additional embodiments, the 5′ flanking sequences are between 10-50 bases immediately upstream of the locus. For example, the 5′ flanking sequences may include 10 bases, 15 bases, 20 bases, 25 bases, 30 bases, 35 bases, 40 bases, 45 bases, or 50 bases immediately upstream of the locus. Each possibility represents a separate embodiment of the present invention.

In some embodiments, the 3′ flanking sequences may include between 1-90 bases immediately downstream of the locus. In some embodiments, the 3′ flanking sequences may include between 5-80 bases immediately downstream of the locus. In some embodiments, the 3′ flanking sequences may include 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 bases immediately downstream of the locus. Each possibility represents a separate embodiment of the present invention.

In some embodiments, the primers may be designed to generate amplification products of between 75-225 bases in length when the locus is intact.

In some embodiments, the method involves simultaneous amplification of more than one target sequence (e.g., at least one marker locus and one control locus) in the same reaction mixture, a process known as multiplex amplification or co-amplification. This process requires simultaneous use of multiple primer pairs. The primers may be designed such that they can work at the same annealing temperature during amplification. In some embodiments, primers with similar melting temperature (Tm) are used in the method disclosed herein. A Tm variation of between about 3°-5° C. is considered acceptable for primers used in a pool.

In some embodiments, all marker and control loci may be amplified in a single reaction mixture. In other embodiments, for example due to technical limitation of a particular machine, the digested DNA sample may be divided into several aliquots, each of which is supplemented with primer pairs for amplification of one or more marker loci and the control locus. Thus, even if a DNA sample is divided into several aliquots, the control locus is amplified in each aliquot, and calculation of signal ratios is performed for the control locus and a marker locus that are amplified together, i.e., from the same aliquot.

In some embodiments, the method may use control constructs comprising non-human DNA sequences in the digestion and amplification step(s). In some embodiments, the control constructs comprise artificial (synthetic) DNA sequences. The control constructs may be used for controlling the digestion and amplification processes, for example, monitoring the efficacy and quality of the digestion and amplification steps. In some embodiments, the control constructs and the DNA sample are digested, by the at least one methylation-sensitive restriction enzyme, at the same time, and optionally, within the same container (e.g. test tube, vial and the like). In some embodiments, the method may include the step of subjecting the control constructs to digestion with the at least one methylation-sensitive restriction enzyme, together with the DNA sample. In some embodiments, the method may include adding the control constructs to the DNA sample and subjecting both to digestion with the at least one methylation-sensitive restriction enzyme.

In some embodiments, a methylation-sensitive restriction endonuclease may be used in the digestion step, together with one or more of the following control constructs: a first control construct comprising a DNA sequence devoid of a recognition sequence of the methylation-sensitive restriction endonuclease, and a second control construct comprising a DNA sequence containing a recognition sequence of the methylation-sensitive restriction endonuclease and being completely unmethylated, such that, the first control construct remains intact, whereas the second control construct is digested completely, or at least partially. In the subsequent amplification steps, primers (and optionally probes) which are specific for the control constructs may be added. Detection of adequate amplification for the first construct concomitant with sufficiently lower amplification for the second construct is indicative of proper DNA digestion.

In some embodiments, detecting adequate amplification for the first construct concomitant with sufficiently lower amplification for the second construct may include detecting a difference of at least 5 cycles between quantification cycles (Cq) of the first and second control constructs in Real-Time PCR. In some embodiments, the difference may be of at least 6 cycles, at least 7 cycles or at least 8 cycles. Each possibility represents a separate embodiment of the present invention.

In some embodiments, the first and second control constructs may include SEQ ID NOs: 17 and 18, respectively. In some embodiments, primers and probes for amplifying and detecting the first control construct may include SEQ ID NO: 19 (forward), SEQ ID NO: 20 (reverse) and SEQ ID NO: 21 (probe). In some embodiment, primers and probes for amplifying and detecting the second control construct may include SEQ ID NO: 22 (forward), SEQ ID NO: 23 (reverse) and SEQ ID NO: 24 (probe).

In some embodiments, amplification of the genomic loci may be carried out using Real-Time PCR (RT-PCR), also known as quantitative PCR (qPCR), in which simultaneous amplification and detection of the amplification products are performed.

In some embodiments, detection of the amplification products in RT-PCR may be achieved using polynucleotide probes, typically fluorescently-labeled polynucleotide probes.

As used herein, “polynucleotide probes” or “oligonucleotide probes” are interchangeable and refer to labeled polynucleotides which are complementary to specific sub-sequences within the nucleic acid sequences of loci of interest, for example, within the sequence of a marker locus or a control locus. In some embodiments, detection is achieved by using TaqMan assays based on combined reporter and quencher molecules (Roche Molecular Systems Inc.). In such assays, the polynucleotide probes have a fluorescent moiety (fluorophore) attached to their 5′ end and a quencher attached to the 3′ end. During PCR amplification, the polynucleotide probes selectively hybridize to their target sequences on the template, and as the polymerase replicates the template it also cleaves the polynucleotide probes due to the polymerase's 5′-nuclease activity. When the polynucleotide probes are intact, the close proximity between the quencher and the fluorescent moiety normally results in a low level of background fluorescence. When the polynucleotide probes are cleaved, the quencher is decoupled from the fluorescent moiety, resulting in an increase of intensity of fluorescence. The fluorescent signal correlates with the amount of amplification products, i.e., the signal increases as the amplification products accumulate.

As used herein, “selectively hybridize to” (as well as “selective hybridization,” “specifically hybridize to,” and “specific hybridization”) refers to the binding, duplexing, or hybridizing of a nucleic acid molecule (such as a primer or a probe) preferentially to a particular complementary nucleotide sequence under stringent conditions. The term “stringent conditions” refers to conditions under which a nucleic acid molecule will hybridize preferentially to its target sequence and to a lesser extent to, or not at all to, other non-target sequences. A “stringent hybridization” in the context of nucleic acid hybridization is sequence-dependent, and differs under different conditions, as known in the art.

Polynucleotide probes may vary in length. In some embodiments, the polynucleotide probes may include between 15-30 bases. In additional embodiments, the polynucleotide probes may include between 25-30 bases. In some embodiments, the polynucleotide probes may include between 20-30 bases, for example, 20 bases, 21 bases, 22 bases, 23 bases, 24 bases, 25 bases, 26 bases, 27 bases, 28 bases, 29 bases, 30 bases. Each possibility represents a separate embodiment of the present invention.

Polynucleotide probes may be designed to bind to either strand of the template. Additional considerations include the Tm of the polynucleotide probes, which should preferably be compatible to that of the primers. Computer software may be used for designing the primers and probes.

As noted above, the methods disclosed herein may involve simultaneous amplification of more than one target sequence (at least one marker locus and one control locus) in the same reaction mixture. In order to distinguish between multiple target sequences that are amplified in parallel, polynucleotide probes labeled with distinct fluorescent colors may be used. In some embodiments, the polynucleotide probes form a fluorophore/quencher pairs as known in the art and include, for example, FAM-TAMRA, FAM-BHQ1, Yakima Yellow-BHQ1, ATTO550-BHQ2 and ROX-BHQ2. In some embodiments, the dye combinations may be compatible to the RT-PCR thermocycler of choice.

In some embodiments, fluorescence may be monitored during each PCR cycle, providing an amplification plot showing the change of fluorescent signals from the probes as a function of cycle number.

In the context of RT-PCR, the following terminology is used:

“Quantification cycle” (“Cq”) refers to the cycle number in which fluorescence increases above a threshold, set automatically by software or manually by the user. In some embodiments, the threshold may be constant for all loci and may be set in advance, prior to carrying out the amplification and detection. In other embodiments, the threshold may be defined separately for each locus after the run, based on the maximum fluorescence level detected for this locus during the amplification cycles.

“Threshold” refers to a value of fluorescence used for Cq determination. In some embodiments, the threshold value may be a value above baseline fluorescence, and/or above background noise, and within the exponential growth phase of the amplification plot.

“Baseline” refers to the initial cycles of PCR where there is little to no change in fluorescence.

Computer software may be used to analyze amplification plots and determine baseline, threshold and Cq.

Following digestion with the at least one methylation-sensitive restriction enzyme, loci in which the CG dinucleotide(s) in the enzyme's recognition site(s) are methylated are amplified with high efficiency, because the DNA molecules are protected from cleavage. The result is relatively low Cq values because detectable amplification products are shown following a relatively small (low) number of amplification cycles. Conversely, loci in which the CG dinucleotide(s) in the enzyme's recognition site(s) are unmethylated are cut more extensively during the digestion step, and thus result in higher Cq values in the amplification and quantification step (i.e., show detectable amplification products following a relatively high number of amplification cycles).

In alternative embodiments, amplification and detection of amplification products may be carried out by conventional PCR using fluorescently-labeled primers followed by capillary electrophoresis of amplification products. In some embodiments, following amplification the amplification products are separated by capillary electrophoresis and fluorescent signals are quantified. In some embodiments, an electropherogram plotting the change in fluorescent signals as a function of size (bp) or time from injection may be generated, wherein each peak in the electropherogram corresponds to the amplification product of a single locus. The peak's height (provided for example using “relative fluorescent units”, rFU) may represent the intensity of the signal from the amplified locus. Computer software may be used to detect peaks and calculate the fluorescence intensities (peak height) of a set of loci whose amplification products were run on the capillary electrophoresis machine, and subsequently the ratios between the signal intensities.

For DNA samples digested with a methylation-sensitive restriction enzyme, e.g., HhaI or HinP1I, loci in which the CG dinucleotide(s) in the enzyme's recognition site(s) are methylated produce a relatively strong signal (higher peak) in the electropherogram. Conversely, loci in which the CG dinucleotide(s) in the enzyme's recognition site(s) are unmethylated produce a relatively weak signal (lower peak) in the electropherogram.

In some embodiments, the fluorescent labels of the primers include any one of fluorescein, FAM, lissamine, phycoerythrin, rhodamine, Cy2, Cy3, Cy3.5, Cy5, Cy5.5, Cy7, FluorX, JOE, HEX, NED, VIC and ROX.

### C. Signal Ratio

The term “ratio” or “signal ratio” as used herein refers to the ratio between the intensities of signals obtained from co-amplification of a pair of genomic loci in a single DNA sample (in the same reaction mixture), particularly co-amplification of a marker locus and a control locus. A signal ratio between a marker locus and a control locus of the present invention, obtained following methylation-sensitive enzymatic digestion and co-amplification, reflects the methylation ratio between these marker and control loci, and represents a “methylation value” according to the present invention.

The term “signal intensity” as used herein refers to a measure reflecting the amount of locus-specific amplification products corresponding to the initial amount of intact copies of the locus. However, the signal intensity may not indicate actual amounts of amplification products/intact loci, and may not involve calculation of any absolute amounts of amplification products/intact loci. Thus, for calculating ratios of amplicon signals, no standard curve or reference DNA may be needed since it is unnecessary to calculate actual DNA concentrations or DNA methylation level per se.

In some exemplary embodiments, amplification and detection of amplification products are carried out by real-time PCR where the signal intensity of a specific locus may be represented by the Cq calculated for this locus. The signal ratio in this case may be represented by the following calculation: 2^{(Cq of control locus-Cq of marker locus)}.

In additional exemplary embodiments, detection of amplification products is carried out by capillary electrophoresis wherein the signal intensity of a specific locus is the number of relative fluorescence units (rfus) of its corresponding peak. The signal ratio may be calculated by dividing the heights of peaks of each marker locus by the height of the peak of a control locus.

In some embodiments, calculating a ratio between signal intensities of the amplification products of a marker locus and a control locus in a DNA sample comprises: (i) determining the signal intensity of the amplification product of the marker locus; (ii) determining the signal intensity of the amplification product of the control locus; and (iii) calculating a ratio between the two signal intensities.

In some embodiments, calculating a ratio between signal intensities of the amplification products of a marker locus and a control locus in the DNA sample comprises determining the Cq for each locus, and calculating the difference between the Cq of the control locus and the Cq of the marker locus. In some embodiments, the calculating further comprises applying the following formula: 2\hat{ }(Cq of control locus-Cq of marker locus).

In some embodiments, calculating a signal ratio may be calculating a plurality of signal ratios, between each marker locus and a control locus.

In some embodiments, a plurality of loci among the loci set forth in SEQ ID NOs: 1-15 are amplified and the methods of the present invention comprise calculating a plurality of signal ratios, e.g., between each of the loci set forth SEQ ID NOs: 1-15 and a control locus, e.g., between the locus set forth in SEQ ID NO: 1 and the control locus, between the locus set forth in SEQ ID NO: 2 and the control locus, and so forth.

In some embodiments, computer software may be used for calculating a ratio between signal intensities of amplification products.

As noted above, a signal ratio between a marker locus and a control locus of the present invention, obtained following methylation-sensitive enzymatic digestion and co-amplification, reflects the methylation ratio between these marker and control loci, and represents a “methylation value” according to the present invention.

### Methylation Analysis Using Methylation-Sensitive Enzymatic Digestion and High-Throughput Sequencing

“High throughput sequencing” (also termed “next generation sequencing”, abbreviated “NGS”) includes sequence determination using methods that determine many (typically thousands to billions) of nucleic acid sequences in parallel. High throughput sequencing generally involves three basic steps: library preparation, sequencing and data analysis. Examples of high throughput sequencing techniques include sequencing-by-synthesis and sequencing-by-ligation (employed, for example, by Illumina Inc., Life Technologies Inc., Roche), nanopore sequencing methods and electronic detection-based methods such as Ion Torrent™ technology (Life Technologies Inc.). High-throughput sequencing include whole genome high-throughput sequencing and target-specific high-throughput sequencing. Each possibility represents a separate embodiment of the present invention.

According to some embodiments of the present invention, following extraction the DNA is subjected to digestion with at least one methylation-sensitive restriction endonuclease as described herein, and subsequently a sequencing library is prepared.

In some embodiments, preparing a sequencing library comprises introducing adapter oligonucleotides, also termed “sequencing adapters”, to DNA fragments, and enriching DNA fragments corresponding to marker loci of interest and optionally one or more control loci. Enrichment of genomic regions of interest may be carried out, for example, using locus-specific PCR, or using capture agents in a solution-phase or a solid-phase hybridization-based process.

The sequencing adapters are oligonucleotides at the 5′ and 3′ ends of each DNA fragment in a sequencing library. Sequencing adapters typically include platform-specific sequences for fragment recognition by a particular sequencer: for example, sequences that enable library fragments to bind to the flow cells of Illumina platforms. Each sequencing instrument typically employs a specific set of sequences for this purpose. The sequencing adapters may include sample indices, which are sequences that enable multiple samples to be sequenced together (i.e., multiplexed) on the same instrument flow cell or chip. Each sample index, typically 6-10 bases, is specific to a given sample library and is used for demultiplexing during data analysis to assign individual sequence reads to the correct sample. Sequencing adapters may contain single or dual sample indexes depending on the number of libraries combined and the level of accuracy desired. Sequencing adapters may be introduced into analyzed DNA fragments by ligation or via PCR. In some embodiments, a 2-step PCR is used, in order to enrich genomic regions of interest and introduce sequencing adapters to the enriched fragments. The first PCR is carried out using primers that contain locus-specific sequences and overhang sequences that introduce a first portion of the sequencing adapters, and the second PCR is carried out using primers that introduce a second portion of the sequencing adapters and optionally sample indices.

A sequencing library is subjected to sequencing to obtain multiple sequence reads. The sequence reads are analyzed using a computer software to determine a read count (copy number) for each locus of interest. The read count of a marker locus reflects the number of methylated copies of this locus that were present in the tested DNA sample (the methylated copies remain intact when the sample is digested with methylation-sensitive restriction endonucleases). Relative copy number may be calculated for a marker locus, for example, with respect to a control locus, as follows:

Relative copy number=Read count of marker locus/Read count of control locus

A read count of a marker locus or alternatively a relative copy number of a marker locus represent “methylation values” according to the present invention.

In some embodiments, analysis of methylation values according to the present invention comprises:
(a) subjecting a DNA sample to digestion with at least one methylation-sensitive restriction endonuclease, thereby obtaining restriction endonuclease-treated DNA;
(b) generating a sequencing library from the restriction endonuclease-treated DNA, the sequencing library comprising DNA fragments corresponding to at least one marker locus as disclosed herein (preferably a plurality of marker loci as disclosed herein) and at least one control locus;
(c) subjecting the sequencing library to high-throughput sequencing and determining a copy number for each of the at least one marker locus and the control locus; and
(d) comparing a ratio between the copy numbers of each of said at least one marker locus and the control locus to at least one reference ratio, to assess the presence of bladder cancer in a subject.

In some embodiments, generating a sequencing library comprises enriching DNA fragments corresponding to the at least one marker locus and the at least one control locus.

### Reference Ratio

The terms “reference ratio” or “reference signal ratio” are used interchangeably and refer to a signal intensity ratio determined in DNA from a known source. A reference ratio for a given pair of marker and control loci may be represented in a number of ways. In some embodiments, the reference ratio for a given pair of loci may be a single ratio. In some embodiments, the reference ratio for a given pair of loci may be a statistic value, such as, the mean value of a large set of reference ratios, obtained from a large set of DNA samples from a known source, e.g., mean value determined in a large group of cancer patients or a mean value determined in a large group of healthy individuals.

In other embodiments, the reference ratio for a given pair of loci may be a plurality of ratios, such as a distribution of ratios determined for this pair of loci in a large set of DNA samples from a known source. In some embodiments, the reference ratio may a reference scale.

In some embodiments, a reference scale for a given pair of loci may include signal ratios measured for this pair of loci in a plurality of DNA samples from the same reference source. For example, a reference scale of reference bladder cancer patients or a reference scale of reference healthy individuals. In other embodiments, a reference scale for a given pair of loci may include signal ratios from both healthy and diseased individuals, i.e. a single scale combining reference ratios from both sources. Generally, when a single scale is used, the values are distributed such that the values from the healthy individuals are at one end of the scale, e.g. below a cutoff, while the values from the cancer patients are at the other end of the scale, e.g., above the cutoff. In some embodiments, a signal ratio calculated for a tested DNA sample from an unknown source may be compared against the reference scale of healthy and cancer reference ratios, and a bladder cancer probability/likelihood score may be assigned to the calculated signal ratio based on its relative position within the scale. In some embodiments, the higher the calculated signal ratio the higher the score assigned thereto, and accordingly the probability with respect to bladder cancer is high.

The terms “bladder cancer reference ratio” or “reference ratio in bladder cancer DNA” interchangeably refer to the signal intensity ratio measured between a given marker locus and a given control locus in DNA from urine samples of bladder cancer patients. The bladder cancer reference ratio represents the signal intensity ratio in bladder cancer DNA, namely, DNA from a cancerous bladder tissue. The bladder cancer reference ratio may be a single ratio, a statistic value or a plurality of ratio (e.g., distribution), as detailed above.

The terms “healthy reference ratio”, “normal reference ratio” or reference ratio in healthy DNA″ interchangeably refer to the signal intensity ratio measured between a given restriction locus and a given control locus in urine samples from normal individuals. A “healthy” or “normal” individual is defined herein as an individual without detectable bladder diseases or symptoms, bladder associated diseases including bladder cancer determined by conventional diagnostic methods. The healthy reference ratio represents the signal intensity ratio in normal bladder DNA, namely, DNA from a normal, non-cancerous bladder tissue. The healthy reference ratio may be a single ratio, a statistic value or a plurality of ratio (e.g., distribution), as detailed above.

In some embodiments, the methods disclosed herein comprise pre-determination of reference ratios from cancerous bladder DNA. In some embodiments, the methods of the present invention comprise pre-determination of reference ratios from normal bladder DNA.

As noted above, a signal ratio may be determined by various methods, including for example measuring peaks following capillary electrophoresis or calculating Cq following real-time PCR. It is to be understood that the reference ratios and ratios measured for a tested sample of an unknown source in order to determine bladder cancer are obtained using the methods disclosed herein.

### Determining the Presence or Absence of Bladder Cancer

In some embodiments, the methods disclosed herein comprise calculating a score for a tested subject based on methylation values and determining whether the score is above or below a predetermined cutoff, wherein detecting that the score is above the cutoff is indicative of a positive likelihood of the presence of bladder cancer in the subject. Detecting that the score is below the cutoff is indicative of a negative likelihood of the presence of bladder cancer in the subject (namely, indicative of a high likelihood of the absence of bladder cancer in the subject). It is to be understood that a negative result in the assays disclosed herein is still considered an assessment/determination of the presence of cancer according to the present invention. In some embodiments, the methods disclosed herein are based on evaluating the signal ratios calculated for DNA from cells of a urine sample of an unknown source compared to reference ratios in order to assess the presence or absence of bladder cancer.

In some embodiments, the calculated signal ratios indicate that the DNA is bladder cancer DNA.

A person of skill in the art would appreciate that the comparison of signal ratios calculated for a tested sample to corresponding reference signal ratios may be performed in a number of ways, using various statistical means.

In some embodiments, comparing a test signal ratio calculated for a given pair of loci to a reference signal ratio comprises comparing the test signal ratio against a single reference value. The single reference value may correspond to a mean value obtained for reference signal ratios from a large population of cancer patients or healthy individuals. In other embodiments, comparing a test signal ratio calculated for a given pair of loci to a reference signal ratio comprises comparing the test signal ratio against a distribution, or scale, of a plurality of reference signal ratios.

Known statistical means may be employed in order to determine whether the signal ratio calculated between a given marker locus and a control locus corresponds to bladder cancer reference ratio or to normal reference ratio. In some embodiments, detecting close approximation of a calculated ratio to bladder cancer reference ratio identifies a subject as a subject having bladder cancer. Conversely, in some embodiments, detecting close approximation of a calculated ratio to normal reference ratio identifies a subject as a subject not having bladder cancer.

In some embodiments, the method comprises comparing a calculated signal ratio to its corresponding bladder cancer reference ratio (i.e., to a signal ratio determined for the same pair of loci in bladder cancer) to obtain a probability score reflecting the likelihood that the calculated signal ratio is a bladder cancer ratio. The better approximation of the calculated signal ratio to the reference ratio, the higher the probability score and accordingly the likelihood that the calculated signal ratio is a bladder cancer ratio. In some embodiments, the probability score is based on the relative position of the calculated signal ratio within the distribution of bladder cancer reference ratios.

In some embodiments, the method comprises comparing a plurality of signal ratios, calculated for a plurality of marker loci with respect to a control locus, to their corresponding bladder cancer references ratios.

In some embodiments, a pattern of signal ratios may be analyzed using statistical means and computerized algorithm to determine if it represents a pattern of bladder cancer or a normal, healthy pattern. Exemplary algorithms include machine learning and pattern recognition algorithms

In some exemplary embodiments, each calculated ratio (for each pair of marker and control locus) may be compared against a scale of reference ratios generated for this pair from a large set of urine samples from both cancer patients and individuals not afflicted with cancer. The scale may represent signal ratios calculated between the pair of marker locus and control locus in a large number of samples from cancer patients and normal individuals. The scale may exhibit a threshold value, also termed hereinafter ‘cutoff’ or ‘pre-defined threshold’, above which are reference ratios corresponding to bladder cancer and below are reference ratios corresponding to healthy individuals.

In some embodiments, the lower ratios, at the bottom of the scale and/or below a cutoff, may be from samples of normal individuals (healthy, i.e., not afflicted with bladder cancer), while the higher ratios at the top of the scale and/or above a predetermined cutoff, may be from the cancer patients. For each ratio (between each marker locus and the control locus), a score may be given based on its relative position on the scale, and the individual scores for each locus are combined to give a single score. In some embodiments, the individual scores may be summed to give a single score. In other embodiments, the individual scores may be averaged to give a single score. In some embodiments, the single score may be used for determining whether the subject is having cancer, where a score above a pre-defined threshold is indicative of bladder cancer.

In some embodiments, a score is a number between 0-100 reflecting the probability that the calculated signal ratio is a bladder cancer ratio, wherein 0 being the lowest probability and 100 being the highest probability. In some embodiments, a threshold score is determined, wherein a score equal to or above which is indicative of bladder cancer.

In additional exemplary embodiments, for each calculated ratio (between each marker locus and the control locus), the probability that it represents bladder cancer DNA may be determined based on comparison to corresponding bladder cancer reference ratio and normal reference ratio, and a score (probability/likelihood score) may be allocated. Consequently, the individual probability scores calculated for each ratio (for each locus) are combined (e.g. summed or averaged) to give a combined score. The combined score may be used for determining whether the subject is having cancer, where a combined score above a pre-defined threshold is indicative of bladder cancer.

Thus, in some embodiments, a threshold, or cutoff, score is determined, above which the subject is identified as having bladder cancer. The threshold score differentiates the population of healthy subjects from the population of non-healthy subject.

In some embodiments, the methods of the present invention comprise providing a threshold score.

In some embodiments, determining the threshold score includes measuring signal ratios in a large population of subjects that are either healthy or have bladder cancer.

In some embodiments, the threshold values are statistically significant values. Statistical significance is often determined by comparing two or more populations, and determining a confidence interval (CI) and/or a p value. In some embodiments, the statistically significant values refer to confidence intervals (CI) of about 90%, 95%, 97.5%, 98%, 99%, 99.5%, 99.9% and 99.99%, while preferred p values are less than about 0.1, 0.05, 0.025, 0.02, 0.01, 0.005, 0.001 or less than 0.0001. Each possibility represents a separate embodiment of the present invention. According to some embodiments, the p value of the threshold score is at most 0.05.

As used herein, the term “about”, when referring to a measurable value is meant to encompass variations of +/−10%, more preferably +/−5%, even more preferably +/−1%, and still more preferably +/−0.1% from the specified value.

In some embodiments, the method further comprises comparing the signal ratio calculated between a given marker locus and a control locus to its corresponding normal bladder reference ratio to obtain a probability score, wherein detecting a low probability score for said ratio with respect to corresponding healthy reference ratio is indicative that the subject has bladder cancer.

In some embodiments, the sensitivity of the methods disclosed herein may be at least 65%. In some embodiments, the sensitivity of the methods may be at least 85%. In some embodiments, a method for detecting or ruling-out high-grade bladder cancer as disclosed herein is characterized by a sensitivity of at least 85%. In additional embodiments, a method for detecting or ruling-out high-grade bladder cancer as disclosed herein is characterized by a sensitivity of at least 87%.

In some embodiments, the “sensitivity” of a diagnostic assay as used herein refers to the percentage of diseased individuals who test positive (percent of “true positives”). Accordingly, diseased individuals not detected by the assay are “false negatives”. Subjects who are not diseased and who test negative in the assay are termed “true negatives.” The “specificity” of the diagnostic assay is one (1) minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In some embodiments, the specificity of the methods disclosed herein is at least 90%. In additional embodiments, the specificity of the methods disclosed herein is at least 95%. In further embodiments, the specificity of the methods disclosed herein is at least 98%, for example between 98-100%.

Management of a Subject with Hematuria and/or Additional Lower Urinary Tract Symptoms Suspicious for Malignancy

Early detection and therapy might improve the prognosis of bladder cancer. However, the high prevalence of hematuria that is not associated with cancer—according to microhematuria guidelines published in 2020 the prevalence of bladder cancer was found to be 0.4% in the low-risk group, 1.0% in the intermediate risk group and 6.3% in the high-risk group—may lead to unnecessary and potentially harmful evaluations. The methods and compositions described herein can be used to increase this diagnostic yield with regard to bladder cancer. According to embodiments of the present invention, hematuria is detected or confirmed in a urine sample of a subject, optionally with at least one additional LUTS. In some embodiments, the methods disclosed herein comprise detecting or confirming hematuria in a urine sample of a subject. A urine sample of the subject is then tested using the methods and systems disclosed herein to determine whether a follow-up bladder cancer confirmatory testing such as cystoscopy is needed.

In addition to the methods described herein for evaluating methylation values of marker loci, clinical evaluation of the patient can provide additional information. This clinical evaluation can be performed prior to evaluating methylation values of marker loci, or can be driven by the methylation value results. Hematuria patients can have various presentations that may include microhematuria, macrohematuria evidenced by red or dark-colored urine or the presence of urinary blood clots. Associated lower urinary tract symptoms (LUTS) may include one or more of: increased urinary frequency, urinary urgency, urge incontinence, nocturia, changes in urine stream, urinary hesitating, straining and dribbling, and incomplete emptying.

In some embodiments, a clinician may perform tests in an effort to differentiate between glomerular causes of hematuria and non-glomerular causes. For example, in some embodiments, clinical evaluation of a subject according to the present invention may include urinalysis. Urinalysis typically includes three components or examinations: physical, chemical, and microscopical. Physical examination includes examining one or more of the volume, color, clarity, odor, and specific gravity. Chemical examination includes measuring one or more of pH, red blood cells, white blood cells, proteins, glucose, urobilinogen, bilirubin, ketone bodies, leukocyte esterase and nitrites. Microscopic examination encompasses the detection of casts, cells, crystals, and/or microorganisms. Presence of 3 or more red blood cells per high power field on urine sediments is defined as microscopic hematuria. Urine appearance, pH, the presence of protein, white blood cells, nitrites, leukocyte esterase, crystals, and casts are also evaluated. A urine specimen with significant white blood cells and positive nitrites and leukocyte esterase suggests urinary tract infection. The presence of excessive proteins with hematuria is indicative of glomerulonephritis.

Urine microscopy examines urine sediments for red blood cell (RBC) morphology. RBC casts may be tested in order to differentiate between glomerular and non-glomerular bleeding as the source of hematuria. Dysmorphic RBCs >25% per high power field are also indicative of glomerulonephritis. Renal parameters such as serum creatinine and urine output may be examined to evaluate the presence or absence of acute kidney injury.

Imaging studies can be in the form of an ultrasound of the kidneys, ureters, and bladder. It can assist in diagnosing, for example, renal stones or bladder or renal mass. It can also detect renal cysts. Abdominopelvic CT scan with or without contrast may be used to detect renal stones and other morphological abnormalities of kidneys. MRI of the abdomen and pelvis may also be used, for example, if CT scan is contraindicated or not diagnostic.

In some embodiments, cystoscopy is performed for a subject with a positive result according to the methylation assay of the present invention in order to further evaluate the presence or absence of a tumor in the bladder of the subject. In some embodiments, urine cytology can be performed to evaluate the presence or absence of malignant cells, but typically it is not a substitute for a cystoscopy.

Computed tomography, with and without contrast, may be used to evaluate patients for bladder cancer, regardless of stage.

Cystoscopic examination under anesthesia (EUA) may be performed to determine whether or not a palpable mass is present, and if present, whether or not it is mobile. An EUA during cystoscopy is effective for identifying locally advanced disease, which may present as gross extravesical extension, invasion of adjacent organs, or pelvic sidewall involvement. If a mass is felt, the bimanual examination may be repeated after the resection to see if it is still present and to differentiate between clinical stage T2 and T3b disease. In general, low-grade, noninvasive tumors are papillary with a narrow stalk. High-grade, invasive tumors frequently can appear sessile, solid, or nodular. Carcinoma in situ (CIS) is a high-grade, noninvasive tumor, which can appear as a flat velvety lesion and can arise in patches. CIS sometimes involves large parts of the urothelial lining.

Fluorescence cystoscopy may also be used, which uses an intravesical photoactive protoporphyrin (such as 5-aminolevulinic acid [5-ALA] and hexyl aminolevulinic acid [HAL]), which accumulates preferentially in neoplastic rather than normal tissue. The photoactive substance enhances the visual difference between normal and neoplastic tissue after illumination with blue light of the appropriate wavelength. The photoactive substance is typically instilled one hour prior to cystoscopy.

When a tumor is identified during cystoscopy, its size and location is documented. A small papillary tumor can be removed either with cold cup biopsy forceps or by transurethral resection of a bladder tumor (TURBT). Narrow Band Imaging may be used to aid in complete resection of bladder tumors. Alternatively, fluorescence cystoscopy may be used.

Despite visually complete resection, there is a high rate of recurrence in patients with non—muscle-invasive bladder cancer (NMIBC). Thus, current American Urologic Association (AUA) guidelines and European Association of Urology (EAU) guidelines each recommend the use of intravesical chemotherapy immediately after TURBT. Several chemotherapeutic agents have shown clinical efficacy in reducing recurrence rates in the post-TURBT setting, including intravesical docetaxel, mitomycin C (MMC), doxorubicin, and epirubicin. Intravesical MMC may also be used following nephroureterectomy. Intravesical Bacillus Calmette-Guérin (BCG) immunotherapy is an established first-line agent in the management of carcinoma in situ (CIS) and high-grade non muscle invasive urothelial carcinoma (UC). These agents may be used in conjunction with one another, for example by administration of MMC prior to scheduled BCG dosing. Intravesical gemcitabine and valrubicin have demonstrated modest activity, with valrubicin being an FDA-approved therapy for the treatment of BCG-refractory CIS. Systemic platinum-based chemotherapy is commonly used for the treatment of locally advanced and metastatic bladder cancer.

Although response rates to traditional therapy is high, many bladder cancer patients suffer from recurrence within 1-5 years. For patients unresponsive to BCG, treatment options are limited; radical cystectomy remains the standard of care. Immune checkpoint inhibitors have become an increasingly used therapeutic option in many solid tumors, including bladder cancers. PD-L1 expression in such tumors may be associated with resistance to intravesical BCG therapy. Thus, the evaluation of patients may include determining the status of PD-L1 expression in bladder tumor tissue, and anti-PD-L1 or anti-PD-1 antibodies may be administered alone or in combination with intravesical BCG or chemotherapy. Since 2016, four checkpoint inhibitor drugs have been approved for bladder cancer: atezolizumab (Tecentriq®), pembrolizumab (Keytruda®), nivolumab (Opdivo®), and avelumab (Bavencio®). Immune checkpoint inhibitors have also demonstrated a higher benefit in heavy CD8 immune cell infiltrated tumors and in tumors with high tumor mutational burden (TMB), such as the case of bladder cancer. Thus, the evaluation of patients may include determining the TMB of bladder tumor tissue. Administration of a checkpoint inhibitor may be by a conventional systemic route, by an intravesical route, or both.

Other biologic therapies that find use in bladder cancer include Enfortumab vedotin, an antibody-drug conjugate targeting nectin-4, which is expressed in many bladder cancers, that contains monomethyl auristatin E. Nadofaragene firadenovec (also known as rAd-IFNa/Syn3) is a replication-deficient recombinant adenovirus that delivers human interferon alfa-2b cDNA into the bladder epithelium. Such agents may be delivered by systemic or intravesical routes, particularly in BCG-unresponsive non-muscle-invasive bladder cancer.

In some embodiments, a score calculated for the subject as disclosed herein based on methylation value(s) of at least one marker locus selected from the group consisting of SEQ ID NOs: 1-15 is determined to be above a pre-defined cutoff. In some embodiments, detecting a score above a pre-defined cutoff identifies primary bladder cancer in the subject. In some embodiments, detecting a score above a pre-defined cutoff determines a positive likelihood for the presence of bladder cancer in the subject, and cystoscopy is performed on the subject with the score above the pre-defined cutoff (namely, with a positive likelihood for the presence of bladder cancer).

In some embodiments, a method for analyzing a urine sample from a human subject with at least one symptom selected from hematuria and lower urinary tract symptoms (LUTS) is provided herein, the method comprising: (a) determining in DNA from cells of the urine sample a methylation value for at least one marker locus selected from the group consisting of SEQ ID NOs: 1-15; and using the methylation value to assess a likelihood that the subject has primary bladder cancer, and, if the likelihood is positive, performing cystoscopy on the subject. If the likelihood is negative, cystoscopy is obviated. In some embodiments, if the likelihood is negative, the subject is allocated for evaluation of the presence or absence of a non-malignant genitourinary condition.

In some embodiments, the methods of the present invention include a step of performing cystoscopy on a subject. As an alternative to a step of performing a cystoscopy, methods of the invention may include a step of scheduling a cystoscopy procedure for the subject, or of allocating a subject for cystoscopy.

In other embodiments, a score calculated for the subject as disclosed herein based on methylation value(s) of at least one marker locus selected from the group consisting of SEQ ID NOs: 1-15 is determined to be below a pre-defined cutoff. In some embodiments, detecting a score below a pre-defined cutoff rules-out bladder cancer in the subject. In some embodiments, detecting a score below a pre-defined cutoff rules-out high-grade bladder cancer in the subject. Thus, in some embodiments, there is provided herein a method for ruling-out bladder cancer. In additional embodiments, there is provided herein a method for ruling-out high-grade bladder cancer. The latter can be performed with sensitivity of at least 85% and exceptionally high specificity of above 98% in subjects presenting with hematuria and/or other lower urinary tract symptoms suspicious of malignancy.

In some embodiments, detecting a score below a pre-defined cutoff rules-out bladder cancer (particularly high-grade bladder cancer) in the subject, and at least one of the following assays is performed on the subject to assess presence of a non-malignant genitourinary condition: serum creatinine measurement, glomerular filtration rate measurement, blood calcium measurement, blood uric acid measurement, urine pH measurement, urine calcium measurement, urine culture, urinary flow test, postvoid residual volume test, renal ultrasound, abdominal ultrasound and prostate ultrasound.

The non-malignant genitourinary condition may include one or more of urinary tract infection, renal infection, renal calculi, bladder calculi, benign prostatic hyperplasia (BPH), renal disease or injury, congenital or acquired anatomic abnormalities, and gynecological disease. Each possibility represents a separate embodiment of the present invention.

In some embodiments, the methods of the present invention comprise detecting a score below a pre-defined cutoff, and performing on the subject repeated urinalysis within a predetermined period of time, for example within 6-12 months (e.g., within 6, 7, 8, 9, 10, 11, 12 months, each possibility represents a separate embodiment of the present invention).

The following are certain preferred embodiments of the invention:

Embodiment 1. A method of performing cystoscopy on a human subject with at least one symptom selected from hematuria and lower urinary tract symptoms (LUTS), comprising:
(a) determining in DNA from cells present in a urine sample of the subject a methylation value for at least one marker locus, and preferably 2, 3, 4, 5, 6, 7, 8, 9, 10, 11, 12, 13, 14, or 15 marker loci, selected from the group consisting of SEQ ID NOs: 1-15;
(b) calculating a score for the subject based on the methylation value;
(c) detecting that the calculated score is above a pre-defined cutoff, thereby determining that primary high-grade bladder cancer is present in the subject; and
(d) performing cystoscopy to identify a location for malignant cells in the bladder in the subject.
In some embodiments, step (d) comprises performing cystoscopy to identify a location for malignant cells in the bladder and to exclude the presence of upper tract urothelial cancer (UTUC) in the subject.

Embodiment 2. A method according to embodiment 1, further comprising biopsy of the malignant cells in the bladder of the subject during the cystoscopy to obtain cancerous tissue.

Embodiment 3. A method according to embodiment 1 or 2, further comprising resection of the malignant cells in the bladder of the subject during the cystoscopy to obtain cancerous tissue.

Embodiment 4. A method according to one of embodiments 1-3, wherein the method further comprises treatment of the bladder lining with an agent that differentially stains malignant cells present in the bladder lining immediately prior to or during cystoscopy.

Embodiment 5. A method according to one of embodiments 1-4, wherein the method further comprises administration of intravesical chemotherapy and/or intravesical immunotherapy during the cystoscopy.

Embodiment 6. A method according to one of embodiments 2 or 3, further comprising distinguishing non-muscle invasive bladder cancer from muscle invasive bladder cancer from the cancerous tissue.

Embodiment 7. A method according to one of embodiments 1-6, wherein the methylation value is calculated separately for each marker locus of SEQ ID NOs: 1-15, and the score is calculated using each of the methylation values calculated.

Embodiment 8. A method of treating bladder cancer in a human subject, comprising:
(a) determining that the subject has at least one symptom selected from hematuria and lower urinary tract symptoms (LUTS);
(b) determining in DNA from cells present in a urine sample of the subject a methylation value for at least one marker locus, and preferably 2, 3, 4, 5, 6, 7, 8, 9, 10, 11, 12, 13, 14, or 15 marker loci, selected from the group consisting of SEQ ID NOs: 1-15;
(c) calculating a score for the subject based on the methylation value;
(d) detecting that the calculated score is above a pre-defined cutoff, thereby determining that primary high-grade bladder cancer is present in the subject;
(e) performing cystoscopy to identify a location for malignant cells in the bladder of the subject and to exclude the presence of upper tract urothelial cancer (UTUC) in the subject; and
(f) administrating intravesical chemotherapy and/or intravesical immunotherapy to the subject.

Embodiment 9. A method according to embodiment 8, further comprising biopsy of the malignant cells in the bladder of the subject during the cystoscopy to obtain cancerous tissue.

Embodiment 10. A method according to embodiment 8 or 9, further comprising resection of the malignant cells in the bladder of the subject during the cystoscopy to obtain cancerous tissue.

Embodiment 11. A method according to one of embodiments 8-10, wherein the method further comprises treatment of the bladder lining with an agent that differentially stains malignant cells present in the bladder lining immediately prior to or during cystoscopy.

Embodiment 12. A method according to one of embodiments 9 or 10, further comprising distinguishing non-muscle invasive bladder cancer from muscle invasive bladder cancer from the cancerous tissue.

Embodiment 13. A method according to one of embodiments 8-12, wherein the subject is administered Bacillus Calmette-Guerin (BCG) as an intravesical immunotherapy.

Embodiment 14. A method according to one of embodiments 8-12, wherein the subject is administered one or more of Nadofaragene firadenovec (Adstiladrin), a checkpoint inhibitor, and Enfortumab vedotin as an intravesical immunotherapy.

Embodiment 15. A method according to one of embodiments 8-12, wherein the subject is administered one or more of valrubicin, mitomycin and gemcitabine as an intravesical chemotherapy.

Embodiment 16. A method of treating bladder cancer in an individual in need thereof, comprising:
intravesically administering a therapeutically effective amount of one or more of BCG, valrubicin, mitomycin and gemcitabine to the individual, wherein the subject has been previously diagnosed with bladder cancer that does not include upper tract urothelial cancer (UTUC), and wherein the previous diagnosis of bladder cancer comprises a combination of (i) identifying cancer histology in bladder tissue obtained by biopsy from the individual during a cystoscopy procedure and (ii) identifying aberrant methylation in at least one marker locus, and preferably 2, 3, 4, 5, 6, 7, 8, 9, 10, 11, 12, 13, 14, or 15 marker loci, selected from the group consisting of SEQ ID NOs: 1-15 present in DNA obtained from cells present in a urine sample obtained from the individual.

### Systems and Kits

In some embodiments, there is provided a kit for assessing the presence or absence of bladder cancer in a subject with at least one symptom selected from hematuria and lower urinary tract symptoms (LUTS) as disclosed herein. In additional embodiments, there is provided a system for assessing the presence or absence of bladder cancer in a subject with at least one symptom selected from hematuria and lower urinary tract symptoms (LUTS) as disclosed herein.

Systems according to the present invention comprise computer processor(s) for performing the assays and/or processing the results e.g., for performing the calculations. In some embodiments, computer-implemented methods are provided herein.

In some embodiments, a system according to the present invention comprises:
DNA extracted from cells of a urine sample of a human subject;
components for carrying out a methylation assay on at least one marker locus hypermethylated in bladder cancer DNA compared to non-cancer DNA selected from the group consisting of SEQ ID NO: 1-15; and
computer software stored on a non-transitory computer readable medium, the computer software directs a computer processor to determine a methylation value for the at least one marker locus based on the methylation assay, and compare the methylation value of each of said at least one marker locus to at least one reference methylation value selected from a cancer methylation value and a non-cancer methylation value, to assess the presence or absence of cancer in the subject according to the methods disclosed herein.

Components for carrying out a methylation assay encompass biochemical components (e.g., enzymes, primers, probes, nucleotides), chemical components (e.g., buffers, reagents), and technical components (e.g., a PCR system, such as a real-time PCR system, and equipment such as tubes, vials, plates, pipettes).

In some particular embodiments, a system according to the present invention comprises:
DNA extracted from cells of a urine sample of a human subject;
at least one methylation-sensitive restriction endonuclease recognizing a sequence within at least one marker locus that is hypermethylated in bladder cancer DNA compared to non-cancer DNA selected from the group consisting of SEQ ID NO: 1-15;
a plurality of primer pairs for co-amplification of the at least one marker locus and a control locus following digestion with the at least one methylation-sensitive restriction endonuclease;
components for detecting amplification products of the at least one marker locus and the control locus; and
computer software stored on a non-transitory computer readable medium, the computer software directs a computer processor to determine a signal intensity for each of the amplification products of the at least one marker locus and the control locus, and compare a ratio between the signal intensities of the amplification products of each of said at least one marker locus and the control locus to at least one reference ratio selected from cancer reference ratio and non-cancer reference ratio, to assess whether cancer is present or absent in the subject according to the methods disclosed herein.

Components for detecting amplification products of the at least one marker locus and the control locus encompass for example fluorescent labels, e.g., in the form of fluorescent primers or fluorescent probes capable of specific hybridization to the amplification products.

In some embodiments, a system according to the present invention prepares and communicates a report to the subject and/or to a healthcare provider of the subject based on the methylation values.

In some embodiments, there is provided herein a system for assessing the presence or absence of bladder cancer in a subject according to the methods disclosed herein, the system comprising a computer software stored on a non-transitory computer readable medium comprising instructions that when executed configure or direct a computer processor to perform the following steps:
(i) receiving a methylation value determined in DNA extracted from cells of a urine sample of the subject for at least one marker locus that is hypermethylated in bladder cancer DNA compared to non-cancer DNA selected from the group consisting of SEQ ID NO: 1-15; and
(ii) comparing the methylation value of each of said at least one marker locus to at least one reference methylation value selected from a cancer methylation value and a non-cancer methylation value, and based on the comparison, outputting an assessment whether cancer is present or absent in the subject.

In some embodiments, the computer software further comprises instructions that when executed configure or direct the computer processor to determine the methylation value of each of said at least one marker locus based on data from a methylation assay.

In some embodiments, a computer software according to the present invention receives as an input raw data of a real-time PCR run. In some embodiments, the computer software directs a computer processor to analyze the real-time PCR data to determine methylation values, e.g., methylation ratios, as disclosed herein.

The computer software includes processor-executable instructions that are stored on a non-transitory computer readable medium. The computer software may also include stored data. The computer readable medium is a tangible computer readable medium, such as a compact disc (CD), magnetic storage, optical storage, random access memory (RAM), read only memory (ROM), or any other tangible medium.

It is understood that the computer-related methods, steps, processes described herein are implemented using software stored on non-volatile or non-transitory computer readable instructions that when executed configure or direct a computer processor or computer to perform the instructions.

Each of the system, server, computing device, and computer described in this application can be implemented on one or more computer systems and be configured to communicate over a network. They all may also be implemented on one single computer system. In one embodiment, the computer system includes a bus or other communication mechanism for communicating information, and a hardware processor coupled with bus for processing information.

The computer system also includes a main memory, such as a random-access memory (RAM) or other dynamic storage device, coupled to bus for storing information and instructions to be executed by processor. Main memory also may be used for storing temporary variables or other intermediate information during execution of instructions to be executed by processor. Such instructions, when stored in non-transitory storage media accessible to processor, render computer system into a special-purpose machine that is customized to perform the operations specified in the instructions.

The computer system further includes a read only memory (ROM) or other static storage device coupled to bus for storing static information and instructions for processor. A storage device, such as a magnetic disk or optical disk, is provided and coupled to bus for storing information and instructions.

The computer system may be coupled via bus to a display, for displaying information to a computer user.

An input device, including alphanumeric and other keys, is coupled to bus for communicating information and command selections to processor. Another type of user input device is cursor control, such as a mouse, a trackball, or cursor direction keys for communicating direction information and command selections to processor and for controlling cursor movement on display.

According to one embodiment, the techniques herein are performed by the computer system in response to the processor executing one or more sequences of one or more instructions contained in main memory. Such instructions may be read into main memory from another storage medium, such as storage device. Execution of the sequences of instructions contained in main memory causes the processor to perform the process steps described herein. In alternative embodiments, hard-wired circuitry may be used in place of or in combination with software instructions.

The term storage media as used herein refers to any non-transitory media that store data and/or instructions that cause a machine to operation in a specific fashion. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus.

In some embodiments, a kit according to the present invention comprises components for carrying out a methylation assay on at least one marker locus as described herein on DNA extracted from cells of a urine sample of a subject.

In some embodiments, the kit comprises at least one methylation-sensitive restriction enzyme; pairs of primers for amplification of at least one marker locus and at least one control locus; and components for detecting amplification products of the at least one marker locus and at least one control locus. In some embodiments, the kit comprises an instruction manual for carrying out the assessment of the presence or absence of cancer as disclosed herein.

In some embodiments, the instruction manual may include instructions for performing the method steps described above.

In some embodiments, the kit or system comprises a single methylation-sensitive endonuclease. In some embodiments, the methylation-sensitive endonuclease is HhaI.

In some embodiments, the kit or system comprises a single methylation-sensitive endonuclease. In some embodiments, the methylation-sensitive endonuclease is HinP1I.

In some embodiments, the kit may further comprise a non-transitory computer readable medium storing a computer software comprising instructions that when executed configure or direct a computer processor to perform the method steps described herein. In some embodiments, the computer software may be a computer software that calculates at least one of signal intensities, signal ratios and marker scores.

In some embodiments, the kit or system comprises: HhaI; primer pairs complementary to at least one marker locus and at least one control locus as described herein; and fluorescent polynucleotide probes complementary to the at least one marker locus and at least one control locus.

In some embodiments, the kit or system comprises: HinP1I; primer pairs complementary to at least one marker locus and at least one control locus as described herein; and fluorescent polynucleotide probes complementary to the at least one marker locus and at least one control locus.

Exemplary primers for amplifying the marker loci set forth in SEQ ID NOs: 1-15 are set forth in SEQ ID NOs: 25-155 as follows (for-forward, rev=reverse):
Locus 1 for.: SEQ ID NOs: 25-29; Locus 1 rev.: SEQ ID NOs: 30-35
Locus 2 for.: SEQ ID NOs: 36-38; Locus 2 rev.: SEQ ID NOs: 39-42
Locus 3 for.: SEQ ID NOs: 43-51; Locus 3 rev.: SEQ ID NOs: 52-61
Locus 4 for.: SEQ ID NOs: 62-64; Locus 4 rev.: SEQ ID NOs: 65-67
Locus 5 for.: SEQ ID NOs: 68-70; Locus 5 rev.: SEQ ID NOs: 71-73
Locus 6 for.: SEQ ID NOs: 74-76; Locus 6 rev.: SEQ ID NOs: 77-79
Locus 7 for.: SEQ ID NOs: 80-85; Locus 7 rev.: SEQ ID NOs: 86-89
Locus 8 for.: SEQ ID NOs: 90-92; Locus 8 rev.: SEQ ID NOs: 93-95
Locus 9 for.: SEQ ID NOs: 96-99; Locus 9 rev.: SEQ ID NOs: 100-102
Locus 10 for.: SEQ ID NOs: 103-106; Locus 10 rev.: SEQ ID NOs: 107-112
Locus 11 for.: SEQ ID NOs: 113-116; Locus 11 rev.: SEQ ID NOs: 117-120
Locus 12 for.: SEQ ID NOs: 121-125; Locus 12 rev.: SEQ ID NOs: 126-130
Locus 13 for.: SEQ ID NOs: 131-135; Locus 13 rev.: SEQ ID NOs: 136-140
Locus 14 for.: SEQ ID NOs: 141-145; Locus 14 rev.: SEQ ID NOs: 146-151
Locus 15 for.: SEQ ID NOs: 152-153; Locus 15 rev.: SEQ ID NOs: 154-155.

Exemplary primers for amplifying the control locus set forth in SEQ ID NO: 16 are set forth in SEQ ID NOs: 156-167 as follows:
Control for.: SEQ ID NOs: 156-161;
Control rev.: SEQ ID NOs: 162-167.

In some embodiments, the kit or system comprises at least one of a first control construct and a second control construct, each comprising non-human/artificial DNA sequences as described above. In some embodiments, the kit comprises both first and second control constructs as described above.

In some embodiments, the kit or system comprises one or more containers filled with at least one nucleotide primer pair. In some embodiments, each nucleotide primer pair included in the kit of the present invention may include primers that are complementary to sub-sequences within a marker locus selected from the marker loci set forth in SEQ ID NOs: 1-15 or to flanking sequences thereof, wherein said each nucleotide primer pair is designed to selectively amplify a fragment of the genome that includes the locus.

In some embodiments, the kit or system may comprise primer pairs for selectively amplifying the combinations of loci described above.

In some embodiments, the kit or system may further comprise nucleotide primer pairs for selectively amplifying the first and second artificial control constructs.

In some embodiments, the kit or system may further include oligonucleotide probes for detecting amplification products of the loci amplified using the primers in the kit. Each oligonucleotide probe may be complementary to a sub-sequence within a locus and may be capable of hybridizing thereto. In some embodiments, the oligonucleotide probes may be fluorescently-labeled.

In some embodiments, the kit or system may further include at least one additional ingredient needed for DNA digestion, loci amplification and detection of amplification products, such as DNA polymerase and nucleotide mix.

In some embodiments, the kit or system may further include suitable reaction buffers for digestion and amplification, and a written protocol for performing bladder cancer identification. The written protocol may comprise instructions for performing any of the steps disclosed herein, including but not limited to, DNA digestion parameters, PCR cycling parameters, signal ratio analysis, and comparison to reference ratios.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1—Specificity of Methylation Markers SEQ ID NOs: 1-15 in a Population of Urology Patients with No Bladder Cancer and Healthy Volunteers

A prospective multicenter study was performed in community urology centers to assess the specificity of methylation markers SEQ ID NOs: 1-15 in a population of urology patients without prior history or evidence of bladder cancer and healthy volunteers. Methylation markers SEQ ID NOs: 1-15 are detailed in Table 1 (previously disclosed in WO 2017/006317, assigned to the Applicant of the present invention). A total of 171 patients were enrolled to the study. Out of this cohort, 53 patients presented with either hematuria (microhematuria or macrohematuria) and/or Lower Urinary Tract Symptoms (LUTS). Table 2 presents the demographics of the cohort.

TABLE 1
Methylation markers

| SEQ | | |
|---|---|---|
| ID | | |
| NO. | Nucleic acid sequence | Description |
| 1 | TTTTTAGGGGTCTGCGCGCTCTCGCC | Position 65676359-65676418 on |
| | TGGCTGCTGGGAAGGAGGGCTGGGA | Chromosome 17, KCNJ2 gene |
| | GGCGCTGAA | |
| 2 | TCCAGGCGCGCCGACCGCTCAAGCG | Position 21958446-21958585 on |
| | CTCCAGGTCCACCCGGCGGAGGGCA | Chromosome 9, CDKN2A gene |
| | GAGAAAGCGCGACCGCGCGGCCCG | |
| | CAGGGTTGCAAGAAGAAAACGAGT | |
| | GTTATATAATGAGTCTCAGTGGTTG | |
| | CTCACAA TGCCAGGCGC | |
| 3 | GGCTGATACCAGAGAGGACCCGGA | Position 336844-336903 on |
| | GCGCGAACCAGAGGTTCGACCTCCA | Chromosome 6, IRF4 gene |
| | G GGCAGCGCAG | |
| 4 | GGGGCGCCAAATGCCCACGTGTTGA | Position 33319507-33319636 on |
| | TGAAACCAGTGAGATGGGAACAGG | Chromosome 21, Olig2 gene |
| | CGGCGGGAAACCAGACAGAGGAAG | |
| | AGCTAGGGAGGAGACCCCAGCCCCG | |
| | GATCCTGGGTCGCCAGGGTTTTCCG | |
| | CGCGCAT | |
| 5 | TCTGAGAAGTGTCCTCCTCGCTCTCT | Position 166502151-166502220 |
| | TATAAAAACAGGACTTGTTGCCGAG | on Chromosome 6, intergenic |
| | GTCAGCGCG CGCATCGAGT | region |
| 6 | ATCAGCGCGAACTATTCGTTTAGTG | Position 896902-897031 on |
| | GCCTTAAAACACCCTGGTTTCACCCT | Chromosome 18, ADCYAPI gene |
| | CAGCTATTTTCAAGTTCCCGTGTGCC | |
| | TGGCACTTTCTCCGTGCGAGAAGCA | |
| | CCGGAGGGTGCGGACGCGCCACAGT | |
| | CTG | |
| 7 | TGCTCTGCGCAGCGTGGAGGGCAAC | Position 32747873-32748022 on |
| | GGGACTGGGAGGCGGCTTCTGCCGC | Chromosome 5, NPR3 gene |
| | CGGGCACTCGCTTCCAGGTGGCTTA | |
| | CGAGGATTCAGACTGTGGGAACCGT | |
| | GCGCTCTTCAGCTTGGTGGACCGCG | |
| | TGGCGGCGGCGCGGGGCGCCAAGCC | |
| 8 | CGCGCAGAACTTTGCGGTGGCGCTT | Position 27949195-27949264 on |
| | AGCGCCTCCTTTGCCCAGACCCTTCC | Chromosome 6, intergenic region |
| | CGCCTTTGCCGCGCCCAGA | |
| 9 | CGTAATCGCCGGTGTAACTCATGTT | Position 27191603-27191672 on |
| | GGCTGGGGGGCCTCCCGGCGCGCGC | Chromosome 7, HOXA9 gene |
| | GGAGAGGCTG GGGTGCGCCC | |
| 10 | CGATTCAGAGGGCCCCGGTCGGAGC | Position 170170302-170170361 |
| | TGTCGGAGATTGAGCGCGCGCGGTC | on Chromosome 16, intergenic |
| | CCGGGATCTC | region |
| 11 | CGCGCGGCAGCCCTCCGTGCGCGCA | Position 30797737-30797876 on |
| | GGCTCGGGTGCGTTGTTCGCGGGGG | Chromosome 15, intergenic region |
| | TGAATTGTGAAGAACCATCGCGGGG | |
| | TCCTTCCTGCTGAGGCCGCGGACAC | |
| | CGTGACCTCGCTGCTCTGGGTCTGC | |
| | AGGGAAACGTAGGAA | |
| 12 | TGACCTTTAGGGGCTGTTACTCTCAG | Position 7936767-7936866 on |
| | ACCAGGCCCAGCAGCACCCGGCGCA | Chromosome 1, intergenic region |
| | TTTACGTCGGATCTGACCCCTGCAA | |
| | GCACCGGCGCGACCGCGCTAGCGG | |
| 13 | AGAACTTCGTGGGCAGGTAAGCGCG | Position 170077565-170077634 |
| | CAGGGCGCGGAGTAAGGATGCGGC | on Chromosome 1, DNM3 gene: |
| | AGTGGGGCGACCCCGCTGCGG | |
| 14 | CTGCCTGCGCTCACTACGGGTTTTTT | Position 1727592-1727661 on |
| | AGTTGGCGCCTTAATGTTTGTAACA | Chromosome 2, PXDN gene |
| | CTTTTAGAGCGCTTGCTCT | |
| 15 | GGCCGGGAAGCGCGGGGTGAGAAA | Position 72919092-72919231 on |
| | GCGAGGTGGGTGGCGAGAGCGTGA | Chromosome 8, MSC gene |
| | GCGCCCCTCTGCTGACCCCGGGGAG | |
| | CGTGGACTACGAGTTGGCGCCCAAG | |
| | TCCAGAATCCGCGCGCACCGCGGTA | |
| | AGCTGCGCCTTTTGAAA | |

*The description refers to position on hg18 genomic build

TABLE 2
Demographics of study participants
Characteristic

| | | |
|---|---|---|
| Age, median (range) | 69 | (27-94) |

| | |
|---|---|
| Sex, M/F % | 110 (64.3%)/61 (35.7%) |
| Smoking history, Non-smoker/Former | 117 (68.4%)/40 |
| smoker/Current smoker | (23.4%)/14 (8.2%) |

Reason for visit:

| | | |
|---|---|---|
| LUTS, n (%) | 41 | (24.0%) |
| Urinary tract infection, n (%) | 28 | (16.4%) |
| Calculi, n (%) | 18 | (10.5%) |
| Hematuria, n (%) | 14 | (8.2%) |
| Other urological conditions^{c}, n (%) | 80 | (46.8%) |
| Healthy volunteer, n (%) | 1 | (0.6%) |
| Concurrent genitourinary cancer, n (%) | 1 | (0.6%)^{a} |
| Concurrent non genitourinary cancer, | 1 | (0.6%)^{b} |
| n (%) | | |

^{a}Vaginal cancer;
^{b}Breast cancer
^{c}Other urological conditions: Benign prostatic conditions, such as: high PSA, BPH, prostatitis, prostate nodules; Benign kidney and urinary tract conditions, such as: hydronephrosis, renal cysts, chronic kidney disease, urethral stricture; Sexual and penile conditions, such as: erectile dysfunction, hematospermia, peyronie's disease, balanitis, infertility, penile lumps and indurations, retrograde ejaculation, low testosterone, herpes and fungal infections; Testicular conditions, such as: epididymitis, testicular hypofunction, hydrocele, testicular pain; Benign gyneco-urological conditions, such as: uterovaginal prolapse, candidiasis of vulva and vagina; Other conditions: hernias, lower back pain.

Urine samples were obtained from the subjects (at least 10 ml of urine from each subject) and a methylation assay was carried out as described in WO 2017/006317, as follows:

First, each urine sample was processed using centrifugation in order to separate the cells (both normal and cancerous, if present) from the urine liquid and create a cell pellet. In particular, each urine sample container was mixed thoroughly such that the sample was homogenous. Next, 10-50 ml urine were transferred to a 50 ml centrifuge tube. For centrifugation the volume was adjusted with PBS 0.01M pH 7.4. Centrifugation was carried out at 1000 g for 10 min at room temperature (15-25° C.). Following centrifugation the supernatant (urine) was discarded and the pellet (cells) was resuspended in the residual urine left in the tube. Following resuspension 40 ml PBS 0.01M pH 7.4 were added to the tube and the pellet was mixed with the PBS by inverting the tube a few times to ensure efficient wash of the cells. Next, a second centrifugation was carried out at 1000 g for 10 min at room temperature (15-25° C.). The PBS was discarded and the pellet was resuspended in the remaining PBS in the tube. The entire pellet volume was then transferred to a 1.5 ml microcentrifuge tube. The required volume for the DNA extraction step is ~400 μl. If the volume was in the range of 400-500 μl, the entire volume was used in the DNA extraction step. If the volume was 500 μl, the tube was centrifuged at 1000 g for 1 min and the supernatant was carefully discarded until reaching approximately 400 μl.

Next, DNA was extracted from the cell pellet using QIAamp blood mini kit (QIAGEN, Hilden, Germany) and subjected to digestion with the methylation-sensitive restriction endonuclease HhaI or HinP1I. The digestion reaction (total volume 120 microliter) included all of the extracted DNA (not quantified) and HhaI or HinP1I in a digestion buffer. The digestion was carried out at 37° C. for 2 hours.

Next, quantitative Real-Time PCR was carried out on the digested DNA samples to amplify in each DNA sample the fifteen marker loci detailed in Table 1 above and a control locus as set forth in SEQ ID NO: 16. The control locus does not contain a recognition sequence of HhaI or HinP1I (does not contain a nucleotide sequence recognized by any of these enzymes, whether methylated or not) and remains intact upon digestion with these enzymes regardless of its methylation status.

More specifically, each digested DNA sample was divided into eight (8) aliquots containing 10 microliters of the digested DNA. Seven aliquots were supplemented with primer pairs for amplification of two marker loci out of the fifteen and the control locus (the control locus is to be amplified in every aliquot). The eighth aliquot was supplemented with a primer pair for amplification of one remaining marker locus and the control locus. Amplicons of between 75 to 225 bases were amplified, each containing one of the 16 loci along with 5′ and 3′ flanking sequences of 5-80 bases. Each amplification reaction (total volume 25 microliter) further contained dNTPs, Taq DNA polymerase and a reaction buffer. To enable detection of amplification products during amplification, fluorescently-labeled polynucleotide probes (one for each locus) were added to the reaction. Real-Time PCR reactions were carried out in an ABI 7500 FastDx instrument with the following PCR program: initial activation of the enzyme of 95° C. for 10 minutes followed by 45 cycles of 15 seconds at 95° C. followed by 1 minute at 60° C.

Following amplification, quantitative PCR plots showing the change of fluorescent signals from the probes as a function of cycle number were analyzed to calculate the quantification cycle (Cq) for each locus. Next, a ΔCq between the Cq of the control locus and the Cq of each marker locus was calculated and used in the following formula:

2^{(Cq of control locus-Cq of marker locus)}.

It is to be understood that the calculation was performed for a marker locus and a control locus that were co-amplified in the same aliquot.

The numerical value obtained for each marker locus with respect to the control locus represents a ratio between the signal intensities of the amplification products of the marker locus and the control locus, and reflects the methylation ratio between the marker locus and the control locus in the DNA sample. Next, a marker score was calculated for each marker locus, the score being the signal ratio normalized in respect to reference ratios such that the highest signal ratio is scored “100” and the lowest signal ratio is scored “0”. Altogether, fifteen (15) marker scores were calculated for each DNA sample. The fifteen individual marker scores obtained for each DNA sample were combined into a single sample score, termed “EpiScore”, which is a number between 0 and 100, reflecting the overall relative methylation level of the DNA sample at the panel of fifteen marker loci. A threshold score was set, above which the DNA sample is classified as positive for bladder cancer. An EpiScore below the threshold classifies the DNA sample as negative for bladder cancer.

A total of 150 patients had a valid methylation assay result, 3 of which were positive, leading to specificity of 98.0% (94.3%-99.6%). In the subset of patients with hematuria or LUTS, specificity was similar 97.8% (88.5%-99.9%). Presence of hematuria, urinary tract infection or calculi did not influence the specificity (p>0.45 by Fisher Exact test). Table 3 presents the specificity by subgroup.

TABLE 3
Specificity for bladder cancer detection by subgroup

| | | Methyl- | | |
|---|---|---|---|---|
| | | ation | | |
| | | assay | | |
| | N | negative | Specificity (CI95%) | |
| All negative patients | 150 | 147 | 98.0% | (94.3%-99.6%) |
| LUTS and/or hematuria | 46 | 45 | 97.8% | (88.5%-99.9%) |
| Urinary tract infection | 27 | 26 | 96.3% | (81.0%-99.9%) |
| Calculi | 15 | 15 | 100.0% | (78.2%-100.0%) |

Another prospective single center study was performed in a urology outpatient clinic to assess the specificity of methylation markers SEQ ID NOs: 1-15 in a population of urology patients without prior history or evidence of bladder cancer presenting with hematuria. A total of 46 patients were enrolled to the study, out of which 31 (67.4%) were males. Table 4 presents the clinical details of the cohort.

TABLE 4
Clinical details of study participants
Characteristic
Diagnosis

| | | |
|---|---|---|
| Calculi, n (%)^{a} | 30 | (65.2%) |
| Benign prostatic hyperplasia (BPH), n (%)^{a} | 12 | (26.1%) |
| Urinary tract infection, n (%) | 4 | (8.7%) |
| Other urological conditions, n (%) | 1 | (2.2%) |

Level of hematuria in the collected urine
sample by Roche Combur-Test ®

| | | |
|---|---|---|
| 1 | 4 | (8.7%) |
| 2 | 1 | (2.2%) |
| 3 | 3 | (6.5%) |
| 4 | 22 | (47.8%) |
| Negative | 16 | (34.8%) |

^{a}one patient had both calculi and BPH

Urine samples were obtained from the subjects and a methylation assay was carried out as described above. A total of 43 patients had a valid methylation assay result, and all were negative, leading to specificity of 100.0% (91.8-100.0%). Table 5 presents the specificity by subgroup.

TABLE 5
Specificity for bladder cancer detection by subgroup

| | | Methylation | |
|---|---|---|---|
| | | assay | |
| | N | negative | Specificity (CI95%) |
| All negative patients | 43 | 43 | 100.0% (91.8-100.0%) |
| Calculi^{a} | 29 | 29 | 100.0% (88.1-100.0%) |
| BPH^{a} | 11 | 11 | 100.0% (71.5-100.0%) |
| Urinary tract infection | 3 | 3 | 100.0% (29.2-100.0%) |

^{a}one patient had both calculi and BPH

### Example 2—Sensitivity of Methylation Markers SEQ ID NOs: 1-15 in Patients with Primary Bladder Cancer

A prospective single center study was performed to assess the sensitivity of methylation markers SEQ ID NOs: 1-15 in patients with primary bladder cancer. A total of 64 patients with newly diagnosed bladder cancer were enrolled to the study, diagnosis was confirmed by pathology for all. Urine samples were collected from each subject prior to resection of the tumor and a methylation assay was carried out as described in Example 1. Median age was 74 (range 43-96) and 56 (87.5%) were males. A total of 62 patients had a valid methylation assay result, 41 of which were positive, leading to overall sensitivity of 66.1% (53.0%-77.7%). Overall, 40 patients were diagnosed with high-grade tumors. The sensitivity for the high-grade tumors was 87.5% (73.2%-95.8%), which was similar across all stages (p=0.5135 by Fisher Exact test). Table 6 presents the overall sensitivity and sensitivity by disease grade and stage.

TABLE 6
Sensitivity in bladder cancer detection,
overall and by disease grade and stage

| | | Methylation | |
|---|---|---|---|
| | N | assay positive | Sensitivity (CI95%) |
| All positive patients | 62 | 41 | 66.1% (53.0%-77.7%) |
| High-grade^{a} | 40 | 35 | 87.5% (73.2%-95.8%) |
| Ta | 18 | 17 | 94.4% (72.7%-99.9%) |
| T1 | 14 | 13 | 92.9% (66.1%-99.8%) |
| T2 | 5 | 4 | 80.0% (28.4%-99.5%) |
| Concomitant CIS^{b} | 15 | 14 | 93.3% (68.1%-99.8%) |
| Low-grade or PUNLMP | 22 | 6 | 27.3% (10.7%-50.2%) |

^{a}Five patients did not have stage information: three low-grade and two high-grade.
^{b}Ten patients did not have concomitant CIS information

In order to calculate negative and positive predictive values (NPV and PPV) of the methylation assay in bladder cancer detection, incidence reported by Woldu et al. (Woldu et al., 2021, J Urol., 205 (5): 1387-1393) was used for the three risk categories defined by the American urological Association (AUA) (Barocas et al., 2020, Microhematuria: AUA/SUFU Guideline. J Urol., 204 (4): 778-786). The calculation used high- and low-grade sensitivities of the methylation assay and the specificities measured and assumed 60% of newly diagnosed cancers were low-grade tumors, and the rest were high-grade, according to literature. The results are presented in Table 7.

TABLE 7
NPV and PPV for bladder cancer detection

| Risk group | Incidence | NPV | NPV | |
|---|---|---|---|---|
| (AUA) | (Woldu) | (all) | (HG) | PPV |
| low | 0.4% | 99.81% | 99.98% | 10% |
| intermediate | 1.0% | 99.52% | 99.95% | 21% |
| high | 6.3% | 96.89% | 99.68% | 64% |

The above table shows that even for the low and intermediate risk groups, the present invention is highly beneficial in reducing the number of unnecessary follow-up tests and in particular cystoscopy:

A pre-test probability of 1% can be converted to a post-test probability of 10% or 21%, for the low and intermediate groups respectively, meaning that 1/10 or ⅕ of cystoscopy tests will show that indeed bladder cancer is present, rather than only 1/100, which substantially cuts down on this invasive procedure. Such effectiveness could allow, for the first time, to select patients to refer to urological evaluation based on a urine test, with a very small risk of missing high-grade disease (0.02-0.32%).

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. A method for determining the presence or absence of primary bladder cancer in a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOS: 1-15;
(b) calculating a score for the subject based on the methylation value; and
(c) detecting that the calculated score is above a pre-defined cutoff, thereby determining a positive likelihood for the presence of primary bladder cancer in the subject.

2. A method for assessing a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is above a pre-defined cutoff, thereby determining a positive likelihood for the presence of bladder cancer in the subject; and
(d) mandating cystoscopy for the subject with the score above the pre-defined cutoff.

3. A method for ruling-out bladder cancer in a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs:
1-15;
(b) calculating a score for the subject based on the methylation value;
(c) detecting that the calculated score is below a pre-defined cutoff, thereby ruling-out bladder cancer in the subject; and
(d) obviating cystoscopy in the subject.

4. A method for assessing a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOS: 1-15;
(b) calculating a score for the subject based on the methylation value;
(c) detecting that the calculated score is below a pre-defined cutoff, thereby ruling-out bladder cancer in the subject; and
(d) performing on the subject at least one assay selected from the group consisting of: serum creatinine measurement, glomerular filtration rate measurement, blood calcium measurement, blood uric acid measurement, urine pH measurement, urine calcium measurement, urine culture, urinary flow test, postvoid residual volume test, renal ultrasound, abdominal ultrasound and prostate ultrasound,
to assess presence of a non-malignant genitourinary condition in the subject.

5. The method of claim 4, wherein the non-malignant genitourinary condition is selected from the group consisting of urinary tract infection, renal infection, renal calculi, bladder calculi, benign prostatic hyperplasia (BPH), renal disease or injury, and congenital or acquired anatomic abnormalities.

6. A method for assessing a human subject with hematuria, the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is below a pre-defined cutoff; and
(d) performing on the subject repeated urinalysis within 6-12 months.

7. The method of claim 6, wherein the subject has one or more lower urinary tract symptoms (LUTS).

8. A method for determining the presence or absence of high-grade primary bladder cancer in a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is above a pre-defined cutoff, thereby determining a positive likelihood for the presence of high-grade bladder cancer in the subject.

9. A method for ruling-out high-grade primary bladder cancer in a human subject with at least one symptom selected from hematuria and lower urinary tract symptoms (LUTS), the method comprising:
(a) determining in DNA from cells of a urine sample of the subject a methylation value for at least one marker locus selected from the group consisting of SEQ ID NOs: 1-15;
(b) calculating a score for the subject based on the methylation value;
(c) detecting that the calculated score is below a pre-defined cutoff, thereby ruling-out high-grade bladder cancer in the subject; and
(d) obviating cystoscopy in the subject.

10. The method of claim 9, further comprising performing on the subject assessment of the presence or absence of a non-malignant genitourinary condition.

11. The method of claim 10, wherein the assessment of the presence or absence of a non-malignant genitourinary condition comprises at least one assay selected from the group consisting of: serum creatinine measurement, glomerular filtration rate measurement, blood calcium measurement, blood uric acid measurement, urine pH measurement, urine calcium measurement, urine culture, urinary flow test, postvoid residual volume test, renal ultrasound, abdominal ultrasound and prostate ultrasound.

12. The method of any one of claims 1-11, wherein the lower urinary tract symptoms (LUTS) comprise at least one symptom selected from: increased urinary frequency, urinary urgency, urge incontinence, nocturia, changes in urine stream, urinary hesitating, straining and dribbling, and incomplete emptying.

13. The method of any one of claims 1-12, wherein the hematuria is microhematuria.

14. The method of any one of claims 1-12, wherein the hematuria is macrohematuria.

15. The method of any one of claims 1-14, wherein the at least one marker locus comprises a plurality of marker loci selected from the group consisting of SEQ ID NOs: 1-15.

16. The method of any one of claims 1-15, wherein the at least one marker locus comprises the locus set forth in SEQ ID NO: 1.

17. The method of claim 16, wherein the at least one marker locus further comprises the locus set forth in SEQ ID NO: 5.

18. The method of claim 17, wherein the at least one marker locus further comprises the locus set forth in SEQ ID NO: 7 and the locus set forth in SEQ ID NO: 11.

19. The method of claim 18, wherein the at least one marker locus further comprises at least one additional marker locus selected from the group of loci set forth in SEQ ID NO: 2, SEQ ID NO: 3, SEQ ID NO: 4, SEQ ID NO: 6, SEQ ID NO: 8, SEQ ID NO: 9, SEQ ID NO: 10, SEQ ID NO: 12, SEQ ID NO: 13, SEQ ID NO: 14 and SEQ ID NO: 15.

20. The method of claim 19, wherein the at least one additional restriction locus comprises the loci set forth in SEQ ID NO: 2, SEQ ID NO: 3, SEQ ID NO: 4, SEQ ID NO: 6, SEQ ID NO: 8, SEQ ID NO: 9, SEQ ID NO: 10, SEQ ID NO: 12, SEQ ID NO: 13, SEQ ID NO: 14 and SEQ ID NO: 15.

21. The method of any one of the preceding claims, wherein the methylation value is determined using one or more analysis methods selected from the group consisting of DNA sequencing, real-time PCR, and array hybridization.

22. The method of any one of the preceding claims, wherein determining the methylation value comprises, for a plurality of potential methylation sites in a plurality of marker loci, quantifying methylated read counts at the potential methylation sites and calculating a methylation value based on the number of methylated read counts.

23. The method of any one of claims 1-21, wherein determining a methylation value for at least one marker locus selected from the group consisting of SEQ ID NOs: 1-15 and calculating a score comprise:
(i) subjecting the DNA from the cells of the urine sample of the subject to digestion with at least one methylation-sensitive restriction endonuclease recognizing a sequence within the at least one marker locus that is hypermethylated in cancer DNA compared to non-cancer DNA, to obtain restriction endonuclease-treated DNA;
(ii) co-amplifying from the restriction endonuclease-treated DNA the at least one marker locus and a control locus, thereby generating an amplification product for each locus;
(iii) determining a signal intensity for each generated amplification product; and
(iv) comparing a ratio between the signal intensities of the amplification products of each of said at least one marker locus and the control locus to at least one reference ratio selected from cancer reference ratio and non-cancer reference ratio, to calculate a score.

24. The method of claim 23, wherein step (i) is performed using a single methylation-sensitive restriction endonuclease.

25. The method of claim 24, wherein the methylation-sensitive restriction endonuclease is HhaI.

26. The method of claim 24, wherein the methylation-sensitive restriction endonuclease is HinP1I.

27. The method of any one of claims 23-26, wherein the control locus is a locus that does not contain a nucleotide sequence recognized by the methylation-sensitive restriction endonuclease.

28. The method of claim 27, wherein the methylation-sensitive restriction endonuclease is selected from HhaI and HinP1I, and the control locus is the control locus is the locus set forth in SEQ ID NO: 16.

29. The method of any one of claims 23-28, wherein step (ii) is performed using real-time PCR.

30. The method of claim 29, wherein step (ii) comprises adding fluorescent probes for assisting in detecting the amplification products of the at least one marker locus and the control locus.

31. The method of claim 20 or claim 30, wherein determining the methylation value comprises, for a plurality of marker loci, determining a ΔCq for each marker locus and calculating a single composite methylation value based on the ΔCq determined for each marker locus.

32. The method of any one of claims 30-32, wherein the ratio between the signal intensities of the amplification products of each of said at least one marker locus and the control locus is calculated by determining the quantification cycle (Cq) for each locus and calculating 2^{(Cq control locus-Cq restriction locus)}.
